(19) United States
(12) Patent Application Publication
(10) Pub. No.: US 2025/0014744 A1
(43) Pub. Date: Jan. 9, 2025

(54) SYSTEMS AND METHODS FOR STREAMING VIDEO FROM A SCANNING SESSION

(71) Applicant: 3SHAPE A/S, Copenhagen K (DK)

(72) Inventors: Frederik JUUL, Copenhagen K (DK); Peter Dahl Ejby JENSEN, Copenhagen K (DK); Anders JELLINGGAARD, Copenhagen K (DK)

(73) Assignee: 3SHAPE A/S, Copenhagen K (DK)

(21) Appl. No.: 18/710,816

(22) PCT Filed: Nov. 17, 2022
(86) PCT No.: PCT/EP2022/082317

§ 371 Date: May 16, 2024

(30) Foreign Application Priority Data
Nov. 17, 2021 (EP) 21208667.2

(51) Int. Cl. G16H 40/67; A61B 1/00; A61B 1/24; A61C 13/34; G06T 17/00; G06T 19/00; H04N 19/136

(52) U.S. Cl. CPC G16H 40/67; A61B 1/00011; A61B 1/0004; A61B 1/00045; A61B 1/00172; A61B 1/24; A61C 13/34; G06T 17/00; G06T 19/00; G06T 2200/24; G06T 2210/41; H04N 19/136

(57) ABSTRACT

The present disclosure relates to a dental scanning system for acquiring scan data of a physical three-dimensional dental object during a scanning session. The dental scanning system includes a scanning device and a first processing device for generating a 3D model. The dental scanning system may further include one or more second processing devices for rendering and/or displaying the 3D model. The dental scanning system is preferably suitable for continuously acquiring, processing, and transmitting images such that a video is streamed during the scanning session. The disclosure further relates to methods related to e.g. transmitting digital images in real-time during a scanning session to one or more external processing devices, and methods for generating a digital three-dimensional (3D) model of a dental object and displaying said 3D model remotely in real-time.

### TECHNICAL FIELD

The present disclosure relates to systems and methods for transmitting images and/or video of a dental object during a dental scanning session. In particular, the disclosure relates to a dental scanning system for acquiring scan data of a physical three-dimensional dental object during a scanning session, and a method of transmitting digital images to one or more external processing devices and/or display devices during a scanning session.

### BACKGROUND

Digital dentistry is increasingly popular and offers several advantages over non-digital techniques. Historically, digital advances had three foci: CAD/CAM systems, dental scanning systems, and practice/patient management systems. Dental scanning system such as intraoral scanners in combination with CAD/CAM systems even made possible delivery of same-day restorations. Practice/patient management software made possible capture of critical data such as patient information, and managing administrative tasks such as tracking billing, and generating reports. Such electronic patient records of patient-centered clinically-oriented information, motivated changes in tracking patients' health, facilitating quality of care assessments, diagnostics and mining data for research, including evaluation of efficiency and efficacy of clinical procedures.

Digital dental scanning systems, both intraoral and laboratory-based, are playing an important role in transforming both restorative and orthodontic dentistry. Real-time imaging using the scanning systems allows for creating three-dimensional digital model of single or multiple teeth, whole arches which may include restorations or implants, opposition arches, occlusion, and surrounding soft tissue or even dentures for edentulous patients. With on-screen display of the three-dimensional digital model, explaining treatment opportunities to patients is simplified. Patients appreciate the more comfortable data-acquisition process. Similarly, dental professionals appreciate the ease and efficiency of using scanning systems. Furthermore, space- and cost-demanding plaster casts/models are replaced by easily archived digital files. Data can be replayed at any time for a variety of different reasons.

CAD/CAM systems are designed to utilize the three-dimensional digital model of patient's teeth to design and fabricate dental restorations and orthodontic appliances ranging from simple inlays to digitally designed and fabricated full dentures, clear aligners, study models, implant-related components, both simple and complex surgical guides. In order to obtain advantages of digital dentistry, different elements such as display, scanning devices, processing units, 3D printers, and other components are operationally connected to one another.

The generation of a digital three-dimensional (3D) model of a dental object, such as a patient's teeth, generally requires high computational power. Therefore, said generation, also referred to as a reconstruction, is typically performed on an external processing device, such as a high-end computer, i.e. a computer considered to have a high processing power. Existing dental scanning systems typically provide a scanning device for acquiring scan data and a (high-end) computer for generating the 3D model. Existing systems typically further feature a display connected to the computer for displaying the 3D model to the dentist and patient or a powerful laptop. Large dental clinics often feature multiple treatment rooms. However, since dental scanning systems are generally considered expensive equipment, oftentimes only one or a few dental scanning systems are acquired for a dental clinic, which implies that the scanning system has to be shared between the treatment rooms. However, it is often cumbersome for the dentist or clinic assistant to move a dental scanning system having a scanning device, a computer, and a display from one treatment room to another.

Therefore, it is desired to develop systems and methods to solve this issue and related issues. It is desirable to obtain a solution that is less costly than present dental scanning systems, and a solution wherein the users (e.g. dentists, surgeons, clinic assistants, etc.) do not have to move the entire dental scanning system from one treatment room to the next for doing scans in succession.

### SUMMARY

The present disclosure solves the above-mentioned challenges by providing a dental scanning system, wherein the processing device configured for generating the digital 3D model is placed at a remote location, i.e. separately from the scanning device. The processing device configured to generate the digital 3D model is referred to herein as the first processing device. The dental scanning system disclosed herein, preferably further comprises one or more second processing devices for displaying images of the 3D model, e.g. on a monitor in the treatment room of the scanning session. An advantage of providing a monitor in the treatment room is that it enables feedback to the dentist during scanning, since a 2D rendition of the digital 3D model may be displayed in real-time during the scanning session. Hence, the dentist is able to see if new scan data is added to the 3D model and he/she is further able to see if enough scan data has been acquired to visualize the desired parts inside the patient's oral cavity. A scanning session may be understood herein as a period of time during which data (such as image data, depth data, color data, or combinations thereof) of a three-dimensional dental object is acquired using the dental scanning system.

Since the computational requirements for displaying images are generally much lower than the requirements of the computer responsible for reconstructing the 3D model, the second processing device(s) may be chosen to be low-powered, lightweight, and relatively cheap devices, compared to the first processing device. A dental clinic having multiple treatment rooms may then acquire multiple such second processing devices, e.g. one for each treatment room, but perhaps only acquire one or a few scanning devices, since these may be shared from one scanning session to the next by use of the presently disclosed systems and methods. If the first processing device is placed at a remote location (e.g. in a server room of the clinic or even in the cloud), it does not need to be moved between the different treatment rooms of the clinic between scanning sessions. The term cloud server or cloud computer may in this context be understood as a remotely located server or computer accessible through the internet.

Accordingly, the presently disclosed system(s) and method(s) solve the problem of having to move large and expensive equipment from room to room. In various embodiments of the disclosed system and method, the first processing device is placed at a remote location, such as provided by a cloud service, which has the additional benefit that software and hardware updates/upgrades are more easily performed, since the updates only need to be performed at one location and on one piece of hardware. Furthermore, the disclosed system and methods reduce the risk of incompatibility issues between different hardware of the dental scanning system, simply because it reduces the amount of hardware equipment (e.g. computers) potentially running different versions of software.

Another related problem is that the reconstruction, i.e. the generation of a digital 3D model of the dental object, is computationally heavy, which implies that a high-end computer is typically needed for this task. A high-end computer may in this context be understood as a computer having high computational power, at least higher than the second processing device(s). Since a high-end computer is typically quite expensive, it is of interest, if a single high-end computer can be common to a plurality of scanning devices, rather than having one high-end computer for each scanning device in each treatment room. However, such a solution will typically imply that two scan sessions cannot run in parallel on the same computer, since the computer will typically only be capable of performing the reconstruction of the digital 3D model associated with one scan session at a time. After the reconstruction, the 3D model has to be rendered in order to be displayed in 2D on a monitor. Presently, the high-end computer is configured for performing both the reconstruction, the rendering, as well as the displaying of the 3D model. A drawback with such a solution is that it will occupy the high-computational computer for the entire scan session, i.e. both for generating the 3D model during the acquisition of the scan data, but also for displaying the 3D model after it has been generated. This implies that a subsequent scan session cannot be initialized before the first scan session has ended. The inventors have realized that by splitting the tasks of reconstructing the model and displaying the model among a first processing device and a second processing device, it is possible to initiate a second scan session even while the 3D model associated with the first scan session is being displayed. Accordingly, in preferred embodiments, the first processing device is utilized during the scanning session to both generate the digital 3D model based on received scan data (or based on received images) and render the digital 3D model. These tasks preferably run continuously as new scan data/images are acquired, and preferably the tasks run in real-time, or perceived real-time to the user. The one or more second processing devices may then advantageously be configured to continuously display the rendered 3D model, preferably similarly in perceived real-time. Once scanning is completed, the 3D model may be sent to the second processing device(s), which may then be configured to render the 3D model after the scanning session is completed. This will liberate the first processing device, such that it is idle and ready to initiate a new scanning session, e.g. using a scanning device in another treatment room.

Common remote desktop solutions such as Splashtop and Teamviewer are capable of streaming the entire desktop session continuously to a remote computer. However, such solutions suffer from the drawback that the high-end computer (referred to herein as the first processing device) would be occupied for the entire scan session including when the scan is done, and the 3D model just needs to be visualized. Another drawback is that the user interface is rendered remotely, which typically implies that the resolution of the user interface is not as good as if it was rendered locally. Yet another drawback is that such a solution would force third-party software to be installed on the client's computer.

According to a first aspect, the present disclosure provides a dental scanning system for acquiring scan data of a physical three-dimensional dental object during a scanning session, the dental scanning system comprising:
a scanning device comprising:
   one or more light projectors configured to generate an illumination pattern to be projected on a three-dimensional dental object during a scanning session; and
   one or more images sensors configured to acquire raw 2D images of the dental object in response to illuminating said object using the one or more light projectors;
a processor configured to generate scan data by processing the raw 2D images, the scan data comprising depth information of the dental object;
a first processing device configured to:
   receive the scan data and/or receive the raw 2D images from the scanning device and subsequently generate scan data by processing the raw 2D images;
   generate a digital 3D model of at least part of the dental object based on the received scan data;
   optionally generate a plurality of digital 2D images of the digital 3D model; and
   transmit the images and/or the 3D model to one or more second processing devices.

In a preferred embodiment, the first processing device is configured to generate a plurality of digital 2D images of the digital 3D model and further configured to encode the digital 2D images in a video encoding format. In this embodiment, the first processing device is configured to transmit the encoded images to the one or more second processing devices. Alternatively, the first processing device is configured to encode the digital 3D model and transmit the encoded 3D model to the one or more second processing devices. The 3D model may also/alternatively be transmitted to the second processing device(s) when the scan session ends, i.e. when scanning is stopped.

In some embodiments, the dental scanning system comprises:
an intraoral scanning device configured to generate scan data associated with the dental object during the scanning session, wherein the scanning device is configured to transmit the scan data to a first processing device;
a first processing device configured to generate a digital 3D representation of the dental object based on the scan data, wherein the first processing device is a remote server or a cloud-based service;
a second processing device configured to:
   render the digital 3D representation; and
   provide a graphical user interface for displaying and controlling the rendering of the digital 3D representation;
wherein the first and second processing devices are configured to communicate with each other via an application programming interface (API), wherein the digital 3D representation may be manipulated and/or updated through one or more user manipulations of the representation via the graphical user interface.

According to a second aspect, the present disclosure relates to a method of transmitting digital images in real-time during a scanning session to one or more external processing devices, the method comprising the steps of:
connect a scanning device to a computer network such as a wireless network, the scanning device being configured to acquire scan data from a three-dimensional dental object during a scanning session;
continuously acquire scan data from the three-dimensional dental object during a scanning session using the scanning device, the scan data comprising a plurality of two-dimensional images and/or point clouds;
continuously transmit the scan data to a first processing device via the computer network;
continuously generate and/or update a digital 3D model of at least part of the dental object based on the received scan data, wherein the generation of the digital 3D model is performed using the first processing device;
continuously generate/render a plurality of digital 2D images of the digital 3D model using the first processing device;
continuously encode the digital 2D images in a video encoding format using the first processing device;
continuously transmit the encoded images to one or more second processing devices; and
continuously decode and display the images, preferably in real-time, using the one or more second processing devices.

In another embodiment of the disclosed method of transmitting digital images, preferably in real-time, during a scanning session to one or more external processing devices, the method comprising the steps of:
connect a scanning device to a computer network such as a wireless network, the scanning device being configured to acquire scan data from a three-dimensional dental object during a scanning session;
continuously acquire scan data from the three-dimensional dental object during a scanning session using the scanning device, the scan data comprising a plurality of two-dimensional images and/or point clouds;
continuously transmit the scan data to a first processing device via the computer network;
continuously generate and/or update a digital 3D model of at least part of the dental object based on the received scan data, wherein the generation of the digital 3D model is performed using the first processing device;
continuously encode the digital 3D model using the first processing device;
continuously transmit the encoded digital 3D model to one or more second processing devices;
continuously decode the encoded digital 3D model using the second processing device(s);
continuously generate a plurality of digital 2D images of the digital 3D model using the second processing device(s); and
continuously display the images in real-time using the second processing device(s).

In yet another aspect, the present disclosure relates to a method of generating a digital three-dimensional (3D) model of a dental object and displaying said 3D model remotely, preferably in real-time, the method comprising the steps of:
receiving scan data of the dental object;
reconstructing a digital three-dimensional (3D) model of at least part of the dental object based on the received scan data, wherein the reconstruction is performed by a first processing device;
rendering a plurality of digital 2D images of the digital 3D model;
encoding the digital 2D images in a video encoding format;
transmitting the encoded 2D images to one or more second processing devices, wherein said second processing devices are located remotely from the first processing device; and
decoding and displaying the 2D images, wherein the decoding and displaying is performed by the one or more second processing devices.

In yet another aspect, the present disclosure relates to a system for displaying images of a digital three-dimensional (3D) model of a dental object, wherein the system comprises:
a first processing device comprising a processor configured to execute machine-readable instructions such that when the machine-readable instructions are executed by the processor, the first processing device is caused to perform:
   receiving scan data of a three-dimensional dental object;
   reconstructing a digital three-dimensional (3D) model of at least part of the dental object based on the received scan data;
   rendering a plurality of digital images of the digital 3D model;
   encoding the digital images in a video encoding format;
   transmitting the encoded images to one or more second processing devices, wherein said second processing devices are located remotely from the first processing device;
one or more second processing devices, each comprising a processor configured to execute machine-readable instructions such that when the machine-readable instructions are executed by the processor, the second processing device(s) are caused to perform:
   decoding the images; and
   displaying the images.

The disclosure further relates to a first computer program configured to generate and/or update a digital 3D model from the scan data. Accordingly, the first computer program may comprise instructions which, when the program is executed by a computer, cause the computer to carry out the step of generating and/or updating a digital 3D model based on received scan data. The first computer program may further comprise instructions which, when the program is executed by a computer, cause the computer to carry out the step of rendering the 3D model. The disclosure further relates to a computer-readable data carrier having stored thereon the first computer program.

The disclosure further relates to a second computer program comprising instructions which, when the program is executed by a computer, cause the computer to carry out the step of generating a graphical user interface for receiving user input. The second computer program may further comprise instructions which, when the program is executed by a computer, cause the computer to carry out the step of rendering and/or displaying the 3D model on a monitor connected to the second processing device(s). The second computer program may further comprise instructions which, when the program is executed by a computer, cause the computer to carry out the step of outputting the digital 2D images of the digital 3D model to a monitor. The disclosure further relates to a computer-readable data carrier having stored thereon the second computer program.

### BRIEF DESCRIPTION OF THE DRAWINGS

FIG. 1 shows an embodiment of a dental scanning system according to the present disclosure.

FIG. 2 shows another embodiment of a dental scanning system according to the present disclosure.

FIG. 3 shows a schematic of a computer.

FIG. 4 shows one example of a dental scanning system according to the present disclosure.

FIG. 5 shows another example of a dental scanning system according to the present disclosure.

FIG. 6 shows yet another example of a dental scanning system according to the present disclosure.

FIG. 7 shows an embodiment of a dental scanning system according to the present disclosure, wherein the dental clinic has multiple treatment rooms.

FIG. 8 shows an embodiment of a method according to the present disclosure.

FIG. 9 shows another embodiment of a method according to the present disclosure.

FIG. 10 shows a decision tree related to the methods disclosed herein.

FIG. 11 shows an embodiment of a dental scanning system according to the present disclosure, wherein some of the key functions processing devices are shown.

FIG. 12 shows another embodiment of a dental scanning system according to the present disclosure.

FIG. 13 shows essentially the same embodiment as the one shown in FIG. 12, however, here the functions are grouped not by processing device but by software module.

FIG. 14 shows a schematic of different software functions and their interactions.

### DETAILED DESCRIPTION

### Dental Object

The three-dimensional dental object may be an intraoral dental object of a patient, said dental object comprising e.g. teeth and/or gingiva of the patient. Such an intraoral dental object may further comprise other objects/materials inside the patient's oral cavity, for example implants or dental restorations. The dental object may only be a part of the patient's teeth and/or oral cavity, since the entire set of teeth of the patient is not necessarily scanned during each scanning session. Examples of dental objects include one or more of: tooth/teeth, implant(s), dental restoration(s), dental prostheses, edentulous ridge(s), and combinations thereof. Alternatively, the dental object may be a gypsum/plaster model representing a patient's teeth.

### Scanning Device

The scanning may be performed by a dental scanning system that may include an intraoral scanning device such as the TRIOS series scanners from 3Shape A/S or a laboratory-based scanner such as the E-series scanners from 3Shape A/S. The scanning device may employ a scanning principle such as triangulation-based scanning, confocal scanning, focus scanning, ultrasound scanning, x-ray scanning, stereo vision, structure from motion, optical coherent tomography OCT, or any other scanning principle. In an embodiment, the scanning device is operated by projecting a pattern and translating a focus plane along an optical axis of the scanning device and capturing a plurality of 2D images at different focus plane positions such that each series of captured 2D images corresponding to each focus plane forms a stack of 2D images. The acquired 2D images are also referred to herein as raw 2D images, wherein raw in this context means that the images have not been subject to image processing.

The focus plane position is preferably shifted along the optical axis of the scanning system, such that 2D images captured at a number of focus plane positions along the optical axis form said stack of 2D images (also referred to herein as a sub-scan) for a given view of the object, i.e. for a given arrangement of the scanning system relative to the object. After moving the scanning device relative to the object or imaging the object at a different view, a new stack of 2D images for that view may be captured. The focus plane position may be varied by means of at least one focus element, e.g., a moving focus lens. The scanning device is generally moved and angled during a scanning session, such that at least some sets of sub-scans overlap at least partially, in order to enable stitching in the post-processing.

The result of stitching is the digital 3D representation of a surface larger than that which can be captured by a single sub-scan, i.e. which is larger than the field of view of the 3D scanning device. Stitching, also known as registration, works by identifying overlapping regions of 3D surface in various sub-scans and transforming sub-scans to a common coordinate system such that the overlapping regions match, finally yielding the digital 3D model. An Iterative Closest Point (ICP) algorithm may be used for this purpose. Another example of a scanning device is a triangulation scanner, where a time varying pattern is projected onto the dental object and a sequence of images of the different pattern configurations are acquired by one or more cameras located at an angle relative to the projector unit.

### Light Projectors

The scanning device comprises one or more light projectors configured to generate an illumination pattern to be projected on a three-dimensional dental object during a scanning session. The light projector(s) preferably comprises a light source, a mask having a spatial pattern, and one or more lenses such as collimation lenses or projection lenses. The light source may be configured to generate light of a single wavelength or a combination of wavelengths (mono- or polychromatic). The combination of wavelengths may be produced by using a light source configured to produce light (such as white light) comprising different wavelengths. Alternatively, the light projector(s) may comprise multiple light sources such as LEDs individually producing light of different wavelengths (such as red, green, and blue) that may be combined to form light comprising the different wavelengths.

Thus, the light produced by the light source may be defined by a wavelength defining a specific color, or a range of different wavelengths defining a combination of colors such as white light. In an embodiment, the scanning device comprises a light source configured for exciting fluorescent material of the teeth to obtain fluorescence data from the dental object.

Such a light source may be configured to produce a narrow range of wavelengths. In another embodiment, the light from the light source is infrared (IR) light, which is capable of penetrating dental tissue.

The light projector(s) may be DLP projectors using a micro mirror array for generating a time varying pattern, or a diffractive optical element (DOF), or back-lit mask projectors, wherein the light source is placed behind a mask having a spatial pattern, whereby the light projected on the surface of the dental object is patterned. The back-lit mask projector may comprise a collimation lens for collimating the light from the light source, said collimation lens being placed between the light source and the mask. The mask may have a checkerboard pattern, such that the generated illumination pattern is a checkerboard pattern. Alternatively, the mask may feature other patterns such as lines or dots, etc.

The scanning device preferably further comprises optical components for directing the light from the light source to the surface of the dental object. The specific arrangement of the optical components depends on whether the scanning device is a focus scanning apparatus, a scanning device using triangulation, or any other type of scanning device. A focus scanning apparatus is further described in EP 2 442 720 B1 by the same applicant, which is incorporated herein in its entirety.

The light reflected from the dental object in response to the illumination of the dental object is directed, using optical components of the scanning device, towards the image sensor(s). The image sensor(s) are configured to generate a plurality of images based on the incoming light received from the illuminated dental object. The image sensor may be a high-speed image sensor such as an image sensor configured for acquiring images with exposures of less than 1/1000 second or frame rates in excess of 250 frames pr. second (fps). As an example, the image sensor may be a rolling shutter (CCD) or global shutter sensor (CMOS). The image sensor(s) may be a monochrome sensor including a color filter array such as a Bayer filter and/or additional filters that may be configured to substantially remove one or more color components from the reflected light and retain only the other non-removed components prior to conversion of the reflected light into an electrical signal. For example, such additional filters may be used to remove a certain part of a white light spectrum, such as a blue component, and retain only red and green components from a signal generated in response to exciting fluorescent material of the teeth.

### Processor

The dental scanning system preferably further comprises a processor configured to generate scan data by processing the two-dimensional (2D) images acquired by the scanning device. The processor may be part of the scanning device, or it may be part of the first processing device. As an example, the processor may comprise a Field-programmable gate array (FPGA) and/or an ARM processor located on the scanning device. The scan data comprises information relating to the three-dimensional dental object. The scan data may comprise any of: 2D images, 3D point clouds, depth data, texture data, intensity data, color data, and/or combinations thereof. As an example, the scan data may comprise one or more point clouds, wherein each point cloud comprises a set of 3D points describing the three-dimensional dental object. As another example, the scan data may comprise images, each image comprising image data e.g. described by image coordinates and a timestamp (x, y, t), wherein depth information can be inferred from the timestamp. The image sensor(s) of the scanning device may acquire a plurality of raw 2D images of the dental object in response to illuminating said object using the one or more light projectors.

The plurality of raw 2D images may also be referred to herein as a stack of 2D images. The 2D images may subsequently be provided as input to the processor, which processes the 2D images to generate scan data. The processing of the 2D images may comprise the step of determining which part of each of the 2D images are in focus in order to deduce/generate depth information from the images. The depth information may be used to generate 3D point clouds comprising a set of 3D points in space, e.g., described by cartesian coordinates (x, y, z). The 3D point clouds may be generated by the processor or by another processing unit. Each 2D/3D point may furthermore comprise a timestamp that indicates when the 2D/3D point was recorded, i.e., from which image in the stack of 2D images the point originates. The timestamp is correlated with the z-coordinate of the 3D points, i.e., the z-coordinate may be inferred from the timestamp. Accordingly, the output of the processor is the scan data, and the scan data may comprise image data and/or depth data, e.g. described by image coordinates and a timestamp (x, y, t) or alternatively described as (x, y, z). The scanning device may be configured to transmit other types of data in addition to the scan data. Examples of data include 3D information, texture information such as infra-red (IR) images, fluorescence images, reflectance color images, x-ray images, and/or combinations thereof.

### Wireless Network Module

The dental scanning system preferably further comprises a wireless network module configured to wirelessly connect the scanning device to a wireless network, such as a wireless local area network (WLAN). The wireless network module may be a part of the scanning device, or it may be a part of an external unit close to the scanning device such as a pod for holding the scanning device. Preferably, the scanning device comprises the wireless network module.

The wireless network module is configured to wirelessly connect the scanning device to a wireless network. The wireless network module may include a chip that performs various functions required for the scanning device to wirelessly communicate with the network, i.e. with network elements that include wireless capability. The wireless network module may utilize one or more of the IEEE 802.11 Wi-Fi protocols/integrated TCP/IP protocol stack that allows the scanning device to access the network. The wireless network module may include a system-on-chip having different types of inbuilt network connectivity technologies. These may include commonly used wireless protocols such as Bluetooth, ZigBee, Wi-Fi, 60 GHz Wi-Fi (WiGig), etc.

A network is to be understood herein as a digital interconnection of a plurality of network elements with the purpose of sending/receiving data between the network elements. The network elements may be connected using wires, optical fibers, and/or wireless radio-frequency methods that may be arranged in a variety of network topologies. Such networks may include any of Personal Area Network (PAN), Local Area Network (LAN), Wireless LAN, Wide Area Network (WAN), or other network types. One or more of the network elements may have access to the internet and network elements may also include a server such as a cloud server. The network elements may include a plurality of components like printers, processing units, displays, modems, routers, computers, servers, storage mediums, identification network elements, etc. As disclosed earlier, these network elements may be connected using one or more of wires, optical fibers or wirelessly, so that at least some of these elements may communicate with one another and directly or indirectly with the scanning device. The scanning device is preferably configured to communicate, using the wireless network module, with at least one other network element via the wireless network.

### Establishing a Wireless Connection

The dental scanning system is preferably configured to establish a wireless connection between the scanning device and any of the first or second processing devices. In some embodiments, the scanning system comprises a scanning device and a first processing device, wherein the two devices are configured to connect to the same wireless network. This may be the case, where the first processing device is located in the clinic. In other embodiments, the first processing device is a remote server or a cloud-based service, i.e. physically located remotely from the clinic and the scanning device. In such a case, the scanning device is typically not connected to the same wireless network as the first processing device. However, in this case, the scanning system preferably comprises a second processing device, which is connected to the same network as the scanning device. In any case, a connection between the scanning device and the first/second processing device needs to be established.

The present inventors have realized many different ways of recognizing the scanning device on the wireless network and establishing a wireless connection between the first or second processing device. In some embodiments, the scanning device is configured to host a network access point for creating an initial connection to the first or second processing device. This allows the scanning device to be recognizable by the first/second processing device. An advantage of this solution is that a connection may be established without relying on further external devices, such as a USB Wi-Fi adapter. In preferred embodiments, a display/monitor is connected to the first or second processing device, e.g. whichever of the two devices are present in the clinic/treatment room. Then, a selection of one or more nearby scanning devices may be presented on the display/monitor, wherein each of the nearby scanning devices visible are hosting a network access point for establishing an initial connection. The selection may be presented as a list in the display, and the list may be sorted according to signal strength. The signal strength may be understood as the strength of the signal broadcasted by the scanning device via the network access point hosted by the scanning device. The wireless connection between the scanning device and the first and/or second processing device may then be established upon selecting a scanning device on the monitor, whereby the scanning device is connected to the wireless network.

In some embodiments, the scanning device comprises a unique identifier, e.g. visible on a surface of the scanning device. The unique identifier may be a serial number e.g. represented as a string of characters, such as letters and/or numbers. As an example, the unique identifier may be represented as a serial number, a QR code, a barcode, or a color code. In some cases, the serial number is provided as input to the system in order to establish a connection between the scanning device and the first/second processing device. In some embodiments, the system is configured to acquire an image of the serial number, e.g. using a camera connected to the dental scanning system. The serial number may then be input automatically to the system, rather than e.g. typing the serial number manually into the system. In some embodiments, the scanning device is configured to transmit the scanner serial number to the first or second processing device using near-field communication (NFC).

In some embodiments, the scanning device comprises a Bluetooth interface for establishing a connection between the first or second processing device based on Bluetooth. In some embodiments, the first or second processing device is configured to search for nearby scanning devices using Bluetooth. Preferably, the first or second processing device is further configured to automatically establish a bidirectional data link between said processing device and the scanning device, wherein the bidirectional data link is based on Bluetooth. The data link allows data/information to be sent to and from the scanning device. The user may authenticate the scanning device to the wireless network via the first or second processing device using the data link/Bluetooth connection. In some embodiments, the scanning system comprises a display/monitor connected to the first or second processing device. Preferably, any nearby scanning devices discovered via Bluetooth are shown on the display/monitor. In some cases, the system is configured such that if the user selects a given scanning device in the display, said scanning device will provide feedback to the user, e.g. in the form of flashing light(s).

In some embodiments, a separate electronic device, such as a smartphone or tablet is utilized for connecting the scanning device to the wireless network. As an example, a Bluetooth connection may be established between the electronic device and the scanning device. This can be achieved if the scanning device features a Bluetooth interface. Then, the scanning device may be visible to the electronic device, e.g. the smartphone. The smartphone may be configured to transfer the Wi-Fi network credentials to the scanning device using said Bluetooth connection. In some embodiments, the separate electronic device is configured to inquire a list of Wi-Fi networks, which is visible to the scanning device. The user may then select a specific Wi-Fi network from said list and enter a password, which is then transferred to the scanning device, whereby it is connected to the network. After transfer of the network credentials and/or after input of a network password, the scanning device may then automatically connect to the wireless network. The electronic device may comprise a software application configured to establish the Bluetooth connection to the scanning device. A list of nearby Bluetooth devices may be presented in the software application, whereby the relevant scanning device may be selected. After selecting a scanning device, in some cases, the user needs to input the password to the wireless network, whereby said password is transferred to the scanning device. In other cases, the connection may be established by transferring a certificate instead of a password.

The scanning device may comprise one or more light sources, e.g. provided as an illumination ring, for providing feedback to the user. The scanning device may further comprise a haptic feedback module for providing haptic feedback, e.g. vibration. The feedback may be correlated with the establishment of the wireless connection, e.g. such that the scanning device provides vibration and/or light upon connecting to the network. The dental scanning system may be further configured to display a list of wireless networks (e.g. Wi-Fi networks), which are visible by the scanning device. The user may then select a given wireless network, whereby a wireless connection may be established, e.g. upon inputting the password of the wireless network. The system may be configured to transmit the password to the scanning device via the Bluetooth data link. Preferably, the scanning device is configured to provide immediate feedback to the user, whether the wireless connection is successfully established or not. The feedback from the scanning device to the first/second processing device may be provided via Bluetooth, e.g. by the aforementioned data link.

In some embodiments, the scanning device is configured for acquiring Wi-Fi credentials of a wireless network by scanning a pattern, such as a QR code or a color code, displayed on a monitor connected to the dental scanning system. Alternatively, the pattern may be provided on a piece of paper. The scanning device may be configured to enter a pattern scanning mode, e.g. by holding a button on the scanning device for a minimum period of time. The Wi-Fi credentials may be encoded in the QR code, such that when the QR code is scanned, the Wi-Fi credentials are automatically transmitted to the scanning device, whereby the scanning device is connected to the wireless network. The Wi-Fi credentials may include the name of the wireless network (SSID) and/or a password (e.g. WPA Key).

### Wireless Network Assessment

In some embodiments, the dental scanning system may be configured to perform a wireless network assessment. The purpose hereof is to assess whether the wireless network fulfills one or more predefined requirements, e.g. to ensure that a stable wireless scanning experience is achieved. As an example, the scanning system may be configured to perform the network assessment immediately after the scanning device is connected to the wireless network. As another example, the network assessment is performed continuously during the scanning session. A variety of properties and/or specifications of the wireless network connection may be measured or assessed during the network assessment. In some cases, the scanning system is configured to generate and display a network assessment report reporting said properties. As an example, the wireless network properties may include one or more of the following: frequency of the current channel being used, signal strength (e.g. measured between the scanning device and a nearby wireless access point), receiving and transmitting bitrate, ping round-trip times, maximum bandwidth, scanning bandwidth, open TCP and UDP ports, Wi-Fi link status, data delay, and packet loss. The properties of the connection may be assessed prior to initiating the scanning and/or during the scanning. As an example, in some embodiments the scanning system is configured to continuously monitor selected properties of the wireless network connection, such as Wi-Fi link status, data delay and packet loss, during the scanning session.

### First Processing Device

According to some embodiments, the dental scanning system comprises a first processing device. In preferred embodiments, the first processing device is configured to:
receive the scan data and/or the raw 2D images from the scanning device via a wireless and/or wired connection;
generate a digital 3D model of at least part of the dental object based on the received scan data;
optionally generate a plurality of digital 2D images of the digital 3D model;
encode the digital 3D model and/or the digital 2D images in a video encoding format; and
transmit the encoded images and/or the encoded 3D model to one or more second processing devices.

The primary objective of the first processing device is to generate the digital 3D model of at least part of the dental object. The first processing device may be a computer, a computer system, a processor, a server, a cloud server, cloud-based services, and/or combinations thereof. As an example, the first processing device may be a single computer, or it may be a plurality of computers connected in a computer cluster. Accordingly, the first processing device may comprise hardware such as one or more central processing units (CPU), graphics processing units (GPU), and computer memory such as random-access memory (RAM) or read-only memory (ROM). The first processing device may comprise a CPU, which is configured to read and execute instructions stored in the computer memory e.g. in the form of random-access memory. The computer memory is configured to store instructions for execution by the CPU and data used by those instructions. As an example, the memory may store instructions, which when executed by the CPU, cause the first processing device to perform the generation of the digital 3D model. The first processing device may further comprise a graphics processing unit (GPU). The GPU may be configured to perform a variety of tasks such as video decoding and encoding, real-time rendering of the 3D model, and other image processing tasks. As an example, the GPU may be configured to manipulate and alter the computer memory to create images in a frame buffer intended for outputting the images to a display.

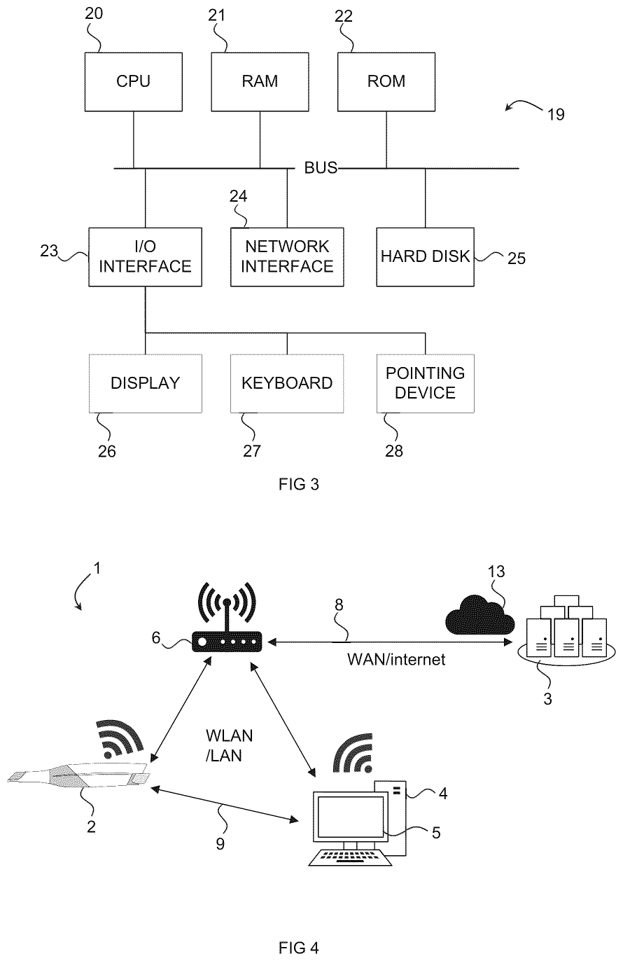

The first processing device may further comprise non-volatile storage in the form of a hard disc drive. The computer preferably further comprises an I/O interface configured to connect peripheral devices used in connection with the computer. More particularly, a display may be connected and configured to display output from the computer. The display may for example display a 2D rendition of the digital 3D model. Input devices may also be connected to the I/O interface. Examples of such input devices include a keyboard and a mouse, which allow user interaction with the first processing device. A network interface may further be part of the first processing device in order to allow it to be connected to an appropriate computer network so as to receive and transmit data (such as scan data and images) from and to other computing devices. In some embodiments, the scan data, e.g. in the form of images or point clouds, are transmitted from the scanning device to the first or second processing device via a wireless network. The CPU, volatile memory, hard disc drive, I/O interface, and network interface, may be connected together by a bus as illustrated in FIG. 3. The computer may further comprise a GPU (not shown) connected to the bus.

In one embodiment, the first processing device is a computer connected to the scanning device, wherein said connection comprise a wired connection, a wireless connection, and/or combinations thereof. The computer may be a remote computer such as a server or a cloud-based server. The term cloud-based may refer to remotely available processing and/or storage services not physically present at the premises (e.g. clinic), where the scanning device is located. In one embodiment, the processor generates scan data such as a plurality of sub-scans. The scan data may comprise image data (i.e. comprising pixel positions (x, y) and intensity) and depth data associated with said image data. The scan data may alternatively comprise image data (i.e. comprising pixel positions (x, y) and intensity) and a timestamp for each image, wherein a depth value can be inferred from said timestamp. Alternatively, the scan data comprises point clouds (i.e. sets of 3D points in space). In case the processor is located on the scanning device, the scanning device is configured to transmit the scan data to the first processing device. Alternatively, the scanning device may be configured to transmit images to an external processor, such as the first processing device, which then generates scan data, e.g. point clouds, from the images. The transmission may occur via a wired connection, a wireless connection, or combinations thereof. For example, the scanning device may be connected to a wireless network and the first processing device may be located on a different network, which may be accessed e.g. through an ethernet connection or the internet. The first processing device is configured to receive, preferably continuously receive, the scan data from the scanning device via a wired and/or wireless connection, or combinations thereof. As an example, the scanning device may be configured to connect to a wireless local area network (WLAN) and it may be further configured to transmit scan data and/or images wirelessly to a router, which relays/routes the scan data/images to the first processing device, e.g. using a wired connection. Hence, the scanning device may comprise a wireless network module, such as a Wi-Fi module, for connecting to a wireless local area network.

In an embodiment, the first processing device is connected to the same network as the scanning device, said network comprising one or more LANs or WLANs. As an example, the scanning device may be located in a treatment room of a clinic and connected to a LAN, and the first processing device may be located in the same clinic and connected to the same LAN. Alternatively, the scanning device may be located in a treatment room of a clinic and connected to a LAN, and the first processing device may be located remotely, i.e. physically separated from the clinic on a different network, such as another LAN or a WAN. This could be the case, if the first processing device is a remote server or a cloud-based processing service. In one embodiment, the first processing device comprises one or more cloud-based processors, e.g. constituting a cloud-based processing cluster. The scanning device and the first processing device may be connected via one or more other network elements, such as gateways, routers, network switches, network bridges, repeaters, repeater hubs, wireless access points, structured cabling, and/or combinations thereof.

In an embodiment, the first processing device is configured to receive scan data, wherein said scan data may comprise images and/or image data (i.e. comprising pixel positions (x, y) and intensity) and a timestamp for each image, wherein a depth value can be inferred from said timestamp. The first processing device is preferably configured to generate processed scan data, such as one or more point clouds based on the images or based on the image data and timestamp(s). Each point cloud comprises a set of data points in space and represents a part of the three-dimensional dental object. The first processing device is preferably configured to further process the scan data, wherein said processing typically comprises the step of stitching overlapping point clouds, whereby an overall point cloud representing the dental object is obtained. Stitching, also known as registration, works by identifying overlapping regions of 3D surfaces in various scan data (e.g. sub-scans) and transforming sub-scans to a common coordinate system such that the overlapping regions match, finally yielding the digital representation, e.g. comprising a single point cloud stitched together from the plurality of point clouds. The stitching typically utilizes best-fit alignment techniques such as an Iterative Closest Point (ICP) algorithm, which aims at minimizing a difference between two clouds of points. The algorithm is conceptually simple and is commonly used in real-time. The algorithm iteratively revises the transformation, i.e. translation and rotation, needed to minimize the distance between the points of two raw scans or sub-scans. The algorithm typically comprises the steps of:
associating points by the nearest neighbor criteria;
estimating transformation parameters using a mean square cost function;
transforming the points using the estimated parameters; and
iterating, i.e. re-associate the points and so on until a termination criteria is met.

The first processing device is preferably configured to further process the digital representation, e.g. by fitting one or more surfaces to the stitched point cloud. The stitching of point clouds and fitting of surfaces may be referred to collectively herein as reconstruction, which has the purpose of generating the three-dimensional digital model of the dental object from the scan data. The reconstruction of the surface of the dental object may be performed using any suitable method and may comprise a triangulation technique. ICP may be used to reconstruct 2D or 3D surfaces from different scan data or sub-scans. Once the digital representation, also referred to herein as the digital 3D model, is generated, the first processing device is preferably configured to update said digital 3D model upon receiving more scan data, e.g. by stitching new point clouds to the overall point cloud and fitting surfaces to the updated model.

In another embodiment, the scanning device is configured to transmit 2D images to the first processing device and said processing device may then be configured to generate point clouds from said images. The scanning device may comprise a processer configured to process the acquired 2D images, whereby processed scan data is generated based on the images. Any of the processed scan data, scan data, and/or images may be transmitted to the first and/or second processing device.

The first processing device is preferably configured to run a first computer program configured to generate and/or update the digital 3D model from the scan data. Accordingly, the first computer program may comprise computer-executable instructions, which when executed, generates and/or updates digital 3D model based on received scan data. The first computer program may further comprise instructions, which when executed, renders the 3D model. The first processing device is preferably configured to execute machine-readable instructions such that when the machine-readable instructions are executed by the first processing device, the first processing device is caused to run the first computer program.

### Rendering

The first processing device is preferably further configured to generate a plurality of digital 2D images of the digital 3D model. This step is also referred to herein as rendering the digital 3D model. Rendering may be understood as the step of generating one or more images from a 3D model by means of a computer program. In other words, rendering is the process of generating one or more images from three-dimensional data. In various embodiments, the rendering is performed by the first computer program, when said application is run/executed by the first processing device. Alternatively, the rendering may be performed by the one or more second processing devices. Hence, the second processing device(s) may be configured to execute a second computer program, which, when executed, performs the step of rendering the 3D model, whereby a plurality of digital 2D images are generated. The digital 2D images differ from the raw 2D images, since the latter is acquired by the image sensor(s) on the scanning device, whereas the former are generated based on the 3D model. The 2D images may be generated by the first processing device or the second processing device(s). The raw 2D images may be used to generate a preview of what is captured inside the patient's oral cavity. Hence, these images display ‘the real world’, whereas the generated digital 2D images represent a specific 2D capture of the digital 3D model of the dental object. The generated 2D images may display the digital 3D model from various angles and zoom-levels. The 2D images may be stored on a computer-readable storage medium readable by the first and/or second processing device. The storage medium may comprise volatile and/or nonvolatile memory. As an example, the 2D images may be stored in a data buffer, such as a DirectX buffer. The 2D images may be generated at a specific, predefined framerate, in order to generate a video comprising said images. The 2D images may be encoded in a video encoding format such as H.264, H.265, or VP8. As an example, 60 images may be generated each second and encoded in a video encoding format, thereby providing a 60 frames per second (FPS) video. Videos encoded with other frame rates may be envisioned. Accordingly, the first processing device may be configured to generate a plurality of digital 2D images of the digital 3D model at a predefined FPS, thereby providing a video. The images and/or video may be transmitted, preferably in real time, to one or more second processing device(s) for decoding and displaying the images/video on a monitor. Said transmission of images/video may also be referred to herein as video streaming or image streaming. Accordingly, the generated images/video may be streamed/transmitted between a first second processing and one or more second processing devices via one or more computer networks and/or the internet.

In the following, an example will be provided to describe at least one way of rendering a 3D model by means of a computer program. First, a virtual camera is defined in the computer program, which is used to define a part of the 3D model that will be projected to a 2D image. The virtual camera may output or define camera data, which may be inputted to a rendering engine for rendering the 3D model. Furthermore, a virtual light source is defined within the computer program, which enables shades on the 3D model. The light source data outputted/defined by the virtual light source may similarly be provided to the rendering engine. The 3D model comprises 3D data such as in the form of 3D meshes comprising vertices and triangles, or in the form of volumetric data. The 3D data may similarly be provided as input to the rendering engine. The rendering engine may form part of the same computer program or be provided in a different computer program, and the rendering engine may be developed according to known standards. The rendering engine is configured to process the input data, such as the camera data, light source data, and 3D data, whereby processed data is generated. As an example, the data prepared for the GPU may include matrices required to project the 3D data to a 2D screen, and buffers comprising the 3D data/geometry that is ready for processing by the GPU. Once the data is made ready, the rendering engine may iterate over the amount of 3D objects that need to be rendered and use relevant techniques, such as DirectX API methods, to provide the data to the GPU. The exact methods of rendering may differ depending on whether the 3D model constitutes a volume comprises a plurality of voxels or if the 3D model constitutes a mesh comprising vertices and triangles. In the first case, the 3D volume is rendered by tracing rays from the pixels in the 2D image until they intersect with the geometry defined by the volume. This is one example of rendering volumetric data, and other known methods for rendering volumetric data may be employed. In the second case, the meshes are rendered by projecting the triangles to the 2D image and filling out the pixels each triangle cover. Other known methods for rendering meshes may be employed.

### Second Processing Device

The dental scanning system preferably further comprises one or more second processing devices for displaying images of the digital 3D model. The second processing device(s) may comprise one or more of: computers, processors, servers, cloud servers, cloud-based services, Internet of Things (IoT) devices, single-board computers (SBC), embedded systems and/or combinations thereof.

Accordingly, the second processing device(s) may comprise hardware such as one or more central processing units (CPU), Graphics Processing Unit (GPU) and computer memory such as random-access memory (RAM) or read-only memory (ROM). The second processing device(s) may comprise a CPU, which is configured to read and execute instructions stored in the computer memory e.g. in the form of random-access memory. The computer memory is configured to store instructions for execution by the CPU and data used by those instructions. The second processing device(s) may further comprise a graphics processing unit (GPU). The GPU may be configured to perform a variety of tasks such as video decoding and encoding, real-time rendering of the 3D model, and other image processing tasks. The computer memory may store instructions, which when executed by the CPU and/or the GPU, cause the second processing device(s) to provide a graphical user interface for receiving user input.

The second processing device(s) may further comprise non-volatile storage e.g. in the form of a hard disc drive. The computer preferably further comprises an I/O interface configured to connect peripheral devices used in connection with the computer. More particularly, a display may be connected and configured to display output from the computer. The display may for example display a 2D rendition of the digital 3D model. Input devices may also be connected to the I/O interface. Examples of such input devices include a keyboard and a mouse, which allow user interaction with the second processing device(s). A network interface may further be part of the second processing device(s) in order to allow it to be connected to an appropriate computer network so as to receive and transmit data (such as scan data and images) from and to other computing devices. The CPU, GPU, volatile memory, hard disc drive, I/O interface, and network interface, may be connected together by a bus.

Each of the second processing device(s) are preferably configured to connect to a display/monitor for displaying the images. Alternatively, the second processing device(s) may comprise an integrated display. In various embodiments, the one or more second processing devices are located remotely from the first processing device. By the term remotely, it may be understood that the first processing device and the second processing device(s) are physically separated and located at different locations. As an example, a dental clinic may feature a plurality of treatment rooms, wherein a second processing device is located in each of said treatment rooms, and the first processing device is located in a separate room of the dental clinic, such as a server room of the dental clinic. In another example, each treatment room of the dental clinic features a second processing device, such as a computer, and the first processing device is located at a remote location from the clinic, e.g. the first processing device is provided as a cloud-based service.

According to some embodiments, the dental scanning system further comprises one or more second processing devices configured to:
receive and decode the encoded images, wherein the encoded images are provided by the first processing device; and
display the decoded images on a monitor connected to or integrated in the second processing device(s).

An advantage of the above-mentioned embodiment wherein the one or more second processing devices are configured to receive and decode encoded images and display the decoded images, is that it does not require so much computational power to decode and display images. As an example, the video encoding/decoding may be performed by a H.264 or H.265 chip set, thereby providing hardware acceleration and consequently low processing demand. Accordingly, in such embodiments, the second processing devices can be selected among low-cost and/or low-powered processing units such as Internet of Things (IoT) devices, single-board computers (SBC), mobile devices such as tablet computers, or other display devices. Preferably, the second processing device(s) is configured to locally (i.e. in the clinic) render a user interface, which may be output to a display connected to the second processing device(s). This may be achieved by a second computer program, configured to be executed by the second processing device(s), wherein a graphical user interface is provided, when the second computer program is executed.

According to other embodiments, the dental scanning system further comprises one or more second processing devices configured to:
receive and decode the encoded digital 3D model, wherein the digital 3D model is provided by the first processing device;
generate digital 2D images of the digital 3D model; and
display the generated images on a monitor connected to or integrated in the second processing device(s).

In the above-mentioned embodiment wherein the one or more second processing devices are configured to receive and decode the encoded digital 3D model and generate digital 2D images of the digital 3D model, the two computational tasks referred to as reconstruction (i.e. the generation of the 3D model) and rendering (i.e. the generation of 2D images of the 3D model) are split between at least two different processing devices, i.e. the first and the second processing device(s). An advantage hereof is that the first processing device is only allocated to perform the heavy computational task of generating the 3D model, whereas other processing devices are rendering the 3D model. This means that the first processing device is occupied for less time compared to the scenario, where it had to perform both tasks. Accordingly, another dentist using another scanning device in another treatment room may then begin scanning as soon as the first scan has ended, since the first processing device is only occupied during the scanning session including a bit of time for post-processing. Accordingly, the first processing device may be configured for generating the 3D model and, during scanning, rendering the 3D model continuously as new scan data is received. Once scanning is complete, i.e. the scanning session has ended, the first processing device is preferably configured to transmit the final generated 3D model, or data allowing a separate processor/computer to build the 3D model, to the one or more second processing devices. In such cases, the second processing device(s) may be configured to render the 3D model after having received the 3D model/3D model data.

The one or more second processing devices are preferably configured to execute machine-readable instructions such that when the machine-readable instructions are executed by the second processing device(s), the second processing device(s) are caused to perform the steps of:
receive and decode the encoded images and/or the 3D model, wherein the encoded images and/or digital 3D model is provided by the first processing device;
optionally generate digital 2D images of the digital 3D model; and
display the decoded images and/or the generated images on a monitor connected to or integrated in the second processing device(s).

In various embodiments, each of the second processing device(s) is a computer connected to the first processing device, wherein said connection is a wired connection, a wireless connection, and/or combinations thereof.

In various embodiments, the second processing device(s) are connected to the same network as the first processing device and/or the scanning device, said network comprising one or more LANs or WLANs. As an example, the scanning device may be located in a treatment room of a clinic and connected to a LAN, and the second processing device(s) may be located in the same treatment room and connected to the same LAN. In this example, the first processing device may be located remotely, i.e. physically separated from the clinic on a different network, such as another LAN or a WAN. The second processing device(s) and the first processing device may be connected via one or more other network elements, such as gateways, routers, network switches, network bridges, repeaters, repeater hubs, wireless access points, structured cabling, and/or combinations thereof. Furthermore, the second processing device(s) may be connected to the scanning device via one or more other network elements as exemplified by the aforementioned list. For example, the scanning device may be configured to provide scan data directly to the second processing device(s). The scan data may be transmitted wirelessly or through a wired connection. As an example, the scan data may be transmitted via Wi-Fi, such as a 2.4 GHz or a 5 GHz Wi-Fi connection. In various embodiments, the scanning device is configured to directly transmit raw images or other data to the second processing device(s). The raw images may be used to provide a 2D preview of the scan during the scanning session. Other relevant data could be motion data for graphical user interface (GUI) navigation. Motion data may be provided in case the scanning device comprises a motion sensor such as a gyroscope or 3D accelerometer. In this case, the scanning device may be used as an input device configured to change the orientation of the rendered 3D model similar to a pointer or mouse. The scanning device may further transmit a 2D image preview data during a scanning session.

In various embodiments, the first and second processing device(s) are configured to connect to each other using a peer-to-peer connection. The peer-to-peer connection may be established via a signaling server configured to send control information between the two devices to determine e.g. the communication protocols, channels, media codecs and formats, and method of data transfer, as well as any required routing information. This process is also known as signaling. The signaling server does not actually need to understand or do anything with the data being exchanged through it by the two peers (here the first and second processing device) during signaling. The signaling server is, in essence, a relay: a common point which both sides connect to knowing that their signaling data can be transferred through it. Accordingly, in various embodiments the first and second processing device(s) are configured to establish the peer-to-peer connection via a signaling server. Any suitable peer-to-peer technology may be utilized for this purpose. As an example, the peer-to-peer connection may be a Web Real-Time Communication (WebRTC) connection. Preferably, the latency of the peer-to-peer connection is low (e.g. below 100 ms) such that the images and/or digital 3D model may be transmitted and received in real-time. In an embodiment, the latency of the peer-to-peer connection is low such as below 200 ms, or below 150 ms, or below 100 ms, preferably below 75 ms. The peer-to-peer connection does not require the first and second processing device(s) to be connected to the same LAN. They may be connected to each other via the internet. Accordingly, in various embodiments the first and second processing device(s) are connected to each other via the internet and/or via one or more computer networks selected among the group of: local area network (LAN), wireless local area network (WLAN), wide area network (WAN), or combinations thereof.

### Second Computer Program

The second processing device(s) are preferably configured to run a second computer program providing a graphical user interface (GUI) for receiving user input, wherein the second computer program is configured to display 2D images of the digital 3D model. The second processing device(s) may be configured to receive 2D image(s), such as raw images, from the scanning device. Such image(s) may be used to provide a pre-view of the dental object in the GUI of the second computer program.

In preferred embodiments, a user may manipulate the digital 3D model generated by the first processing device using the GUI of the second computer program running on the second processing device(s). Hence, in such embodiments the graphical user interface is rendered locally on the second processing device(s). This is in contrast to existing remote desktop solutions, where the user interface is rendered remotely. At least one advantage of rendering the GUI locally is that the resolution of the GUI is better compared to if it is rendered remotely, which will provide a better experience for the user. Another advantage is that the task of rendering the GUI is provided by a separate processing device, such that the first processing device is liberated from this task, whereby this device can be used for other scanning tasks. The GUI may provide a plurality of options, whereby user manipulations of the 3D model may be performed. As an example, such user manipulations may be selected from the group of: rotate the model, move the view parallel to the view plane (pan), zoom in/out on the model, change texture of the model, change colors of the model, add/change fluorescent colors, and/or combinations thereof. Further user manipulations relating to a 3D model of a dental object may include: trim, lock, marked preparations, clearing the scan, manual bite alignment result, adjust for contacts, and/or combinations thereof. The former group of user manipulations relate to the visualization/rendering of the 3D model, i.e. the ability of the user to control/change the visualization of the model. The latter group of user manipulations relate to interactions with the 3D model. Such manipulations need to be provided to the reconstruction engine (part of the first computer program) running on the first processing device. The user manipulations may be specified in an application programming interface (API). According to various embodiments, the first and second computer programs are configured to communicate with each other via an application programming interface (API). The first processing device is preferably configured to receive user input/user manipulations via one or more application programming interface (API) calls. The user input and/or user manipulations may be provided in the second computer program as mentioned above.

The second computer program may be configured to receive data associated with the digital 3D model or the digital 3D model itself, and then display the data associated with the digital 3D model directly or render the 3D model, i.e. generate 2D images of the 3D model. The images may then be displayed in the second computer program, which may be displayed on a monitor connected to or integrated in the second processing device(s). When a user interacts with the 3D model through the GUI in the second computer programs, then in some cases these interactions/manipulations need to be provided as instructions to the first computer program running on the first processing device. This could be the case if the manipulations require that the 3D model is rebuilt/updated. In this case, the user manipulations may be specified in an application programming interface (API) as described above. In other cases, the user manipulations may only relate to rendering the model, which may be performed locally by the second processing device(s).

In an example illustrating a typical scanning session, the user (e.g. a dentist) utilizes a scanning device such as an intraoral 3D scanner to image the inside of a patient's oral cavity, whereby a plurality of raw 2D images are obtained. In some embodiments, the intraoral 3D scanner comprises a processor to process the 2D images, whereby scan data is obtained. The scan data typically comprises depth information, which is associated with the images. Alternatively, the scan data may comprise other data, such as timestamp(s), which can be used to infer the depth from the images. The scan data may comprise other information as well. The scan data is then continuously transmitted, preferably in real-time, to the first processing device during the scanning session. The first processing device then continuously builds a digital three-dimensional (3D) model of the scanned object inside the patient's oral cavity. As new scan data is received by the first processing device, the 3D model is continuously updated and re-built based on the new scan data. In some embodiments, the 3D model is rendered by the first processing device, i.e. 2D images are generated, wherein said 2D images show a rendition of the 3D model. These 2D images may then, continuously, be encoded in a video encoding format in order to compress the size of the images and create a video stream. The video encoding format may be any encoding suitable for generating a video stream, e.g. H.264, H.265, or VP8. The encoded 2D images may then be transmitted to the one or more second processing device(s) at a predefined frame rate, such as a frame rate of at least 30 frames per second, preferably at least 60 frames per second. The second processing device(s) may then display the transmitted images at the predefined frame rate, whereby a video is displayed continuously and approximately simultaneous (i.e. with a low latency) with the generation/updating of the 3D model during the scanning session. Preferably, the entire process (imaging, reconstruction, rendering, transmission, displaying) happens in perceived real-time, i.e. the end-user may experience that the model is being generated and displayed at the same time as the user is scanning new parts of the (dental) object. In reality, of course a small lag/latency is present, however it is preferred to have a very small latency. In preferred embodiments of the disclosed systems/methods, the latency is below 100 ms, more preferably below 75 ms, even more preferably below 50 ms. Ideally, this is the case regardless of the physical location of the scanning device, the first and the second processing devices. In other embodiments, it is the 3D model (or the data associated with said 3D model), which is being transmitted. In that case, the second processing device(s) is preferably configured to generate the 2D images of the model, i.e. perform the rendering step before the images can be displayed. In various embodiments, the 3D model (or data associated with said model) is transmitted by the first processing device to the second processing device(s) once scanning is complete and stopped, i.e. when the scanning session terminates. In such embodiments, the second processing device(s) are configured to render the 3D model based on the received 3D model and/or data associated with said 3D model.

### Microservices

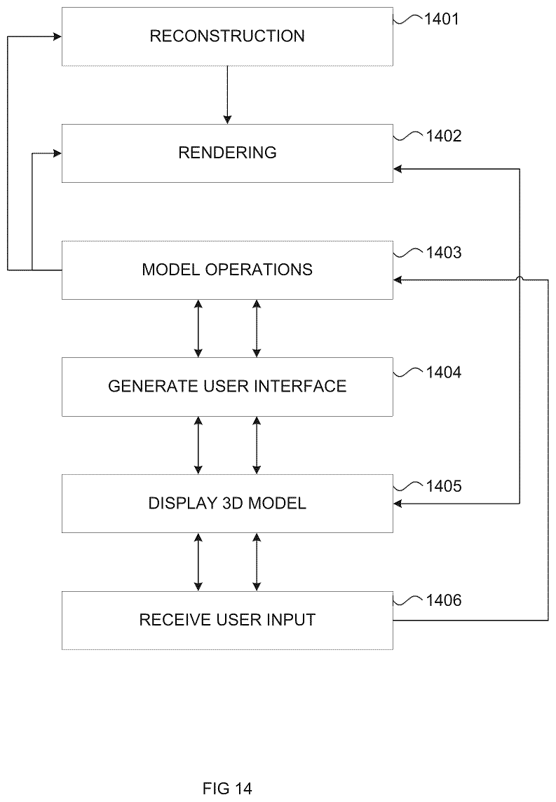

The methods described herein may be performed, or realized, wholly or partly by means of one or more computer programs such as the first and/or second computer program. Accordingly, some steps of the disclosed method(s) may be provided by a first computer program, and other steps may be provided by a second computer program, etc. The different computer programs may also be referred to herein as microservices. Hence, the computer programs may collectively form a microservice architecture. This is explained further in relation to FIG. 14. In the following, a plurality of different microservices are given as an example. The microservices may be seen as being provided by the steps of one or more computer-implemented methods, each method comprising the steps of:
receiving raw 2D images and/or scan data of a dental object, said images comprising image data, from a scanning device; and
filtering the image data of the raw 2D images, wherein outliers and noisy data is removed from the images, whereby filtered images are generated; and/or
segmenting the images into desirable and undesirable areas, e.g. displaying tissue, using artificial intelligence, whereby segmented images are generated; and/or
orienting and/or correcting the orientation of images/scan data in relation to previously received scan data in a scanning session, whereby aligned images/scan data are/is generated; and/or
filtering undesired data from images based on determining whether object(s) between the scanning device and the dental object are temporary artifact(s) and not part of the dental object, whereby marked images are generated; and/or
merging aligned images/scan data into coherent 3D geometry by maintaining a surface describing the entire scan and averaging this surface with new incoming scan data, whereby fused images/scan data are/is generated; and/or
rendering 2D image(s) from either fused images/scan data or aligned images/scan data, whereby 2D image(s) are generated which may be output to a monitor for displaying the image(s); and/or
generating a three-dimensional (3D) mesh from a plurality of aligned images or aligned scan data, or from a plurality of fused images/fused scan data, whereby a 3D mesh is generated representing the dental object; and/or
optimizing the global accuracy by minimizing error(s) in the alignment between the images received from the scanning device.

Accordingly, the present disclosure relates to one or more computer programs, each computer program comprising instructions which, when the computer program is executed by a computer, cause the computer to carry out the steps of the aforementioned computer-implemented method.

Notice that the aforementioned computer-implemented method comprises a plurality of method steps separated by ‘and/or’. Hence, in some embodiments, the computer-implemented method comprises only a few of said method steps, which may then be carried out by a computer executing a first computer program. In another embodiment, the computer-implemented method comprises other steps, which may then be carried out by a computer executing a second computer program, etc. Numerous combinations of method steps exist which may be provided by a plurality of computer programs.

As an example, one of the disclosed computer-implemented methods may comprise the steps of:
receiving raw 2D images and/or scan data of a dental object, said images comprising image data, from a scanning device; and
filtering the image data of the raw 2D images, wherein outliers and noisy data is removed from the images, whereby filtered images are generated.

This method may be provided by the execution of a third computer program, said computer program being an example of a microservice.

As another example, one of the disclosed computer-implemented methods may comprise the steps of:
receiving raw 2D images and/or scan data of a dental object, said images comprising image data, from a scanning device; and
orienting and/or correcting the orientation of images/scan data in relation to previously received scan data in a scanning session, whereby aligned images/scan data are/is generated.

This method may be provided by the execution of fourth computer program, said computer program being an example of a microservice.

### DETAILED DESCRIPTION OF DRAWINGS

FIG. 1 shows an embodiment of a dental scanning system 1 according to the present disclosure. The scanning system comprises a scanning device 2, a first processing device 3, a second processing device 4, and a monitor 5 connected to the second processing device. In this embodiment, the scanning device is an intraoral 3D scanner for acquiring images inside the oral cavity of a patient. The scanning device and the second processing device is connected to a wireless LAN, which is established by a router 6. The scanning device, second processing device, and the monitor may be located in a treatment room 7. The scanning device is configured to transmit first data 8, such as sub-scans, to the first processing device, and second data 9, such as raw images for 2D previews and gyro data, to the second processing device. The first processing device comprises a first computer program 10 comprising a reconstruction module 11 configured to generate a 3D model from the sub-scans received from the scanning device, and a rendering module 12 configured to render the 3D model, i.e. generate 2D images of the 3D model. The first processing device may be located on a separate computer network, and it may be located in the cloud 13. The scanning device and the first processing device may be connected via the internet and via the router. The second processing device comprises a second computer program 14 comprising a model operations module 15 and a graphical user interface module 16 for generating a graphical user interface (GUI) to be output to a monitor 5. A user may perform operations on the 3D model via the GUI, and the model operations module is then configured to transmit said operations/instructions to the reconstruction module and/or the rendering module depending on the type of operations. The computer system comprising the first and second processing devices preferably implements an application programming interface (API) 17, such that the first and second computer program are connected to each other via the API. The model operations may then be transmitted via one or more API calls.

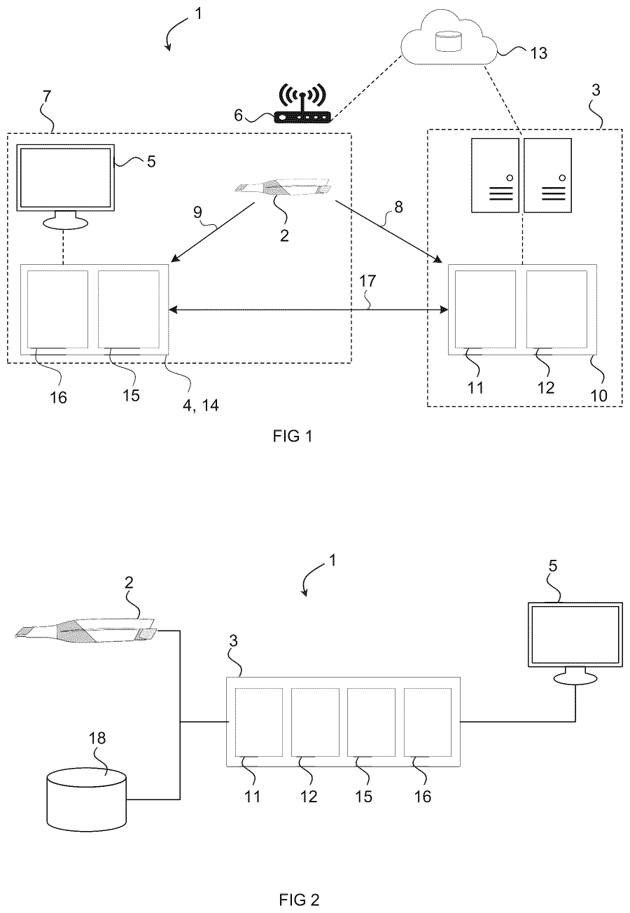

FIG. 2 shows an embodiment of a dental scanning system 1 according to the present disclosure. The scanning system comprises a scanning device 2, a first processing device 3, and a monitor 5 connected to the first processing device. In this embodiment, the first processing device comprises all the software functions described in relation to the embodiment shown in FIG. 1. Hence, the first processing device 3 is configured to execute both the first and second computer program (10, 14), which may comprise several software modules/functions as explained earlier and also shown in FIG. 14. The first and second computer programs may form part of the scanning software application. Other software applications may form part of a larger software ecosystem 18, which may be connected to the scanning software application.

FIG. 3 shows a schematic of a computer 19. The first and/or second processing devices (3, 4) may constitute or comprise a computer according to this figure. The computer 19 comprises a central processing unit (CPU) 20, which is configured to read and execute instructions stored in a computer memory, which may take the form of volatile memory such as random-access memory (RAM) 21. The computer memory stores instructions for execution by the CPU and data used by those instructions. For example, the instructions may relate to the methods disclosed herein, such as the generation of the 3D model or the rendering of said 3D model. The computer further comprises non-volatile storage, e.g. in the form of a hard disk 25. The computer further comprises an I/O interface 23 to which other devices may be connected. Such devices may include display(s) 26, keyboard(s) 27, and pointing device(s) 28 e.g. a mouse. The display 26 is configured to display a 2D rendition of the digital 3D model of the dental object. A user may interact with the computer via a keyboard 27 and/or a mouse 28. A network interface 24 allows the computer to be connected to an appropriate computer network so as to receive and transmit data from and to other computing devices. The CPU, computer memory (RAM/ROM), hard disk, I/O interface, and network interface are connected together by a bus.

FIG. 4 shows an embodiment of a dental scanning system 1 according to the present disclosure. The scanning system comprises a scanning device 2, a first processing device 3, a second processing device 4, and a monitor 5 connected to the second processing device. The scanning device and the second processing device are connected to a local area network (LAN), such as a wireless local area network (WLAN). The LAN/WAN may be established by an access point and/or a router 6 such as a wireless router. The scanning device 2 may be a 3D intraoral scanner. The scanning device may be configured to transmit first data 8 to the first processing device via the router 6, and it may be further configured to transmit second data 9 wirelessly to the second processing device 4, optionally via the router 6. In this embodiment, the first processing device is a cloud-based service 13, e.g. comprising a processing cluster in the cloud, i.e. as a remote service. The first processing device is present on a wide area network (WAN), which may be reached via the internet.

FIG. 5 shows an embodiment of a dental scanning system 1 according to the present disclosure. The scanning system comprises a scanning device 2, a first processing device 3, a second processing device 4, and a monitor 5 connected to the second processing device. The scanning device and the second processing device are connected to a local area network (LAN) e.g. via wired connection such as an ethernet connection. The LAN may be established by a router 6. The scanning device is configured to transmit data to the second processing device via the wired connection. The transmitted data may comprise first data 8, such as sub-scans, and second data 9, such as raw images or motion data. The first processing device 3 is a cloud-based service 13, e.g. comprising a processing cluster. The first and second processing devices are connected to each other via a router 6 connected to the internet.

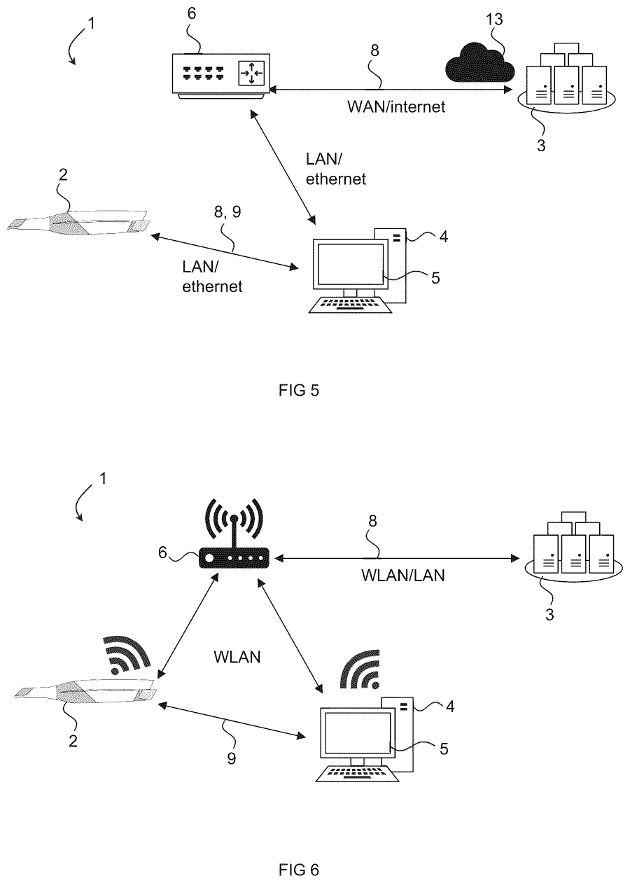

FIG. 6 shows an embodiment of a dental scanning system 1 according to the present disclosure. This embodiment is largely similar to the embodiment shown in FIG. 4. However, in this embodiment the first processing device 3 is a computer or server, which is connected to either a WLAN or a LAN, wherein the WLAN may be the same WLAN as the one that the scanning device 2 and second processing device 4 are connected to. Hence, the first processing device 3 does not need to be a cloud-based service. Instead, it could be a computer located at the premises of the clinic. The first processing device is connected to the router 6 either wirelessly or by a wired connection.

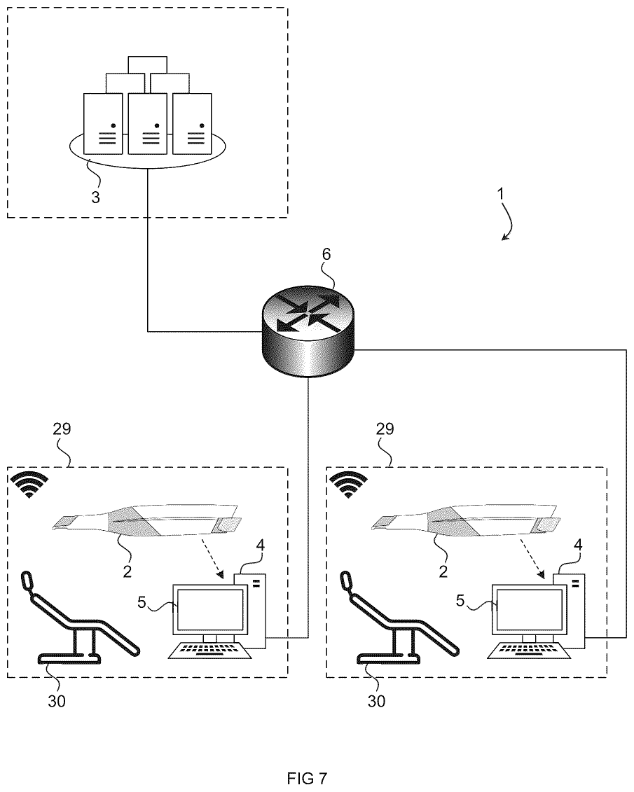

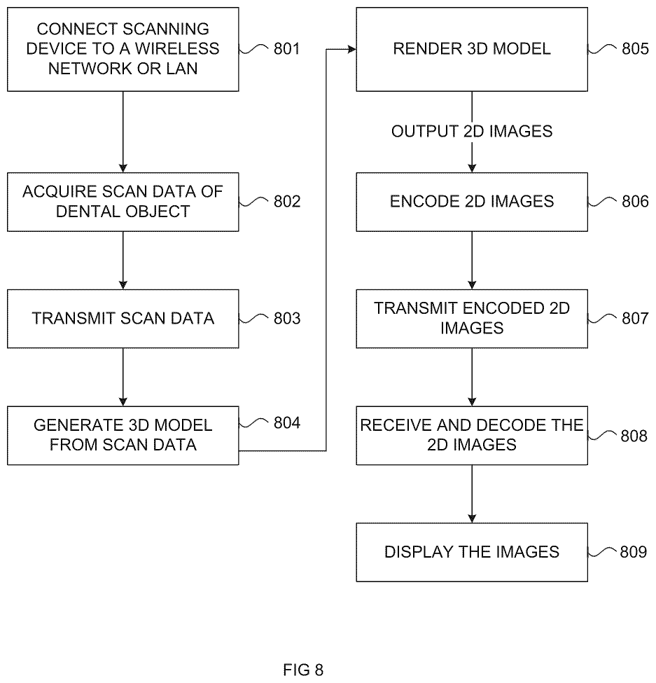

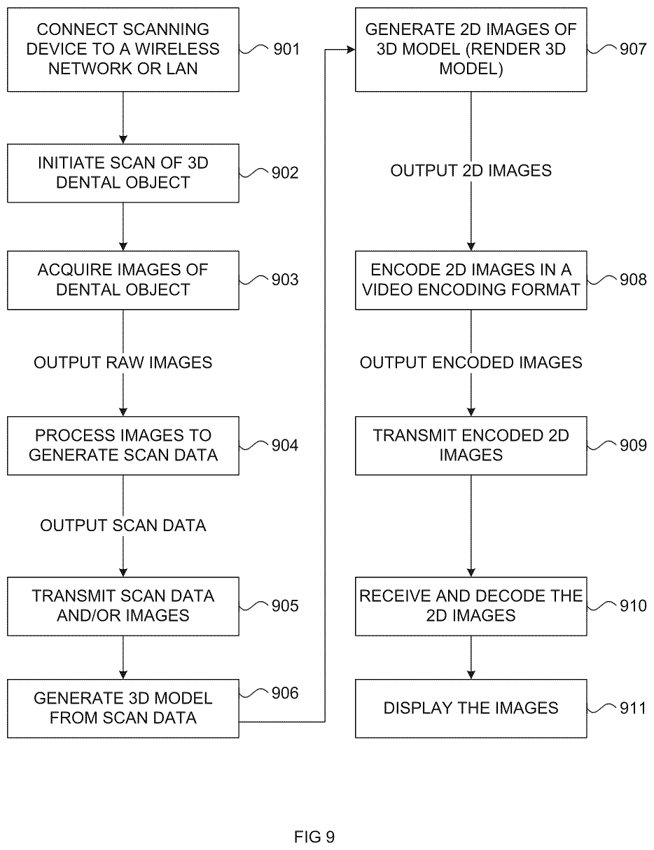

FIG. 7 shows an embodiment of a dental scanning system 1 according to the present disclosure. According to this embodiment, the dental scanning system comprises a first processing device 3, e.g. constituting a processing cluster, and one or more second processing devices 4 connected to the first processing device e.g. via a switch or router 6. As an example, the dental clinic may feature a plurality of treatment rooms 29, wherein the second processing devices 4 are distributed among them, such that each treatment room features a second processing device (here exemplified as a computer). Each treatment room may further feature a scanning device 2 configured to transmit data to the second processing device. Alternatively, one scanning device 2 may be shared between the different treatment rooms 29. Preferably, the second processing device is configured to automatically recognize a nearby scanning device and connect to it wirelessly e.g. upon user confirmation. The devices (scanning device and second processing device) may be connected to the same wireless network, illustrated by the Wi-Fi symbol in the treatment rooms. Each treatment room 29 may further comprise a dental chair 30 for the patient. The first processing device 3 may be configured to execute a first computer program comprising instructions to generate a 3D model based on received scan data. The second processing devices may be configured to execute a second computer program. The second computer program may comprise instructions, which when executed, generates a graphical user interface for receiving user input. The second computer program may further comprise instructions, which when executed, renders and/or displays the 3D model on a monitor 5 connected to the second processing device(s) 4.

FIG. 8 shows an embodiment of a method according to the present disclosure. The method comprises the steps of connecting a scanning device, e.g. a 3D intraoral scanner, to a computer network such as a wireless network or a local area network (801). Then, a scanning session is initiated, wherein scan data of a dental object is acquired (802). The scan data is then transmitted (803) via the computer network to a first processing device, e.g. a computer or processing cluster, configured to generate a 3D model from the scan data (804). Then, the 3D model is rendered (805), whereby a plurality of 2D images are generated. The 2D images are encoded (806) and transmitted (807) to one or more second processing devices configured to decode (808) and display (809) the images. The method can run continuously such that the 3D model is updated continuously as scan data is acquired, and the displayed images updates accordingly. The transmitted stream of 2D images may constitute a video stream, e.g. transmitted with a predefined frame rate such as 60 Hz.

FIG. 9 shows an embodiment of a method according to the present disclosure. This embodiment is largely similar to the method described in relation to FIG. 8, however this particular method comprises more steps/more details. First, a scanning device is connected to a wireless network or a local area network (901). Then, a scan is initiated (902), wherein images are acquired (903). These images are then processed to generate scan data (904), which is transmitted (905), possibly along with the (raw) images. Then a 3D model is generated (906) based on the scan data. The 3D model is then rendered (907) to generate a plurality of 2D images which are outputted. The 2D images of the 3D model are then encoded (908) in a video encoding format (e.g. H.264, H.265, VP8), whereby a video stream is generated. The encoded 2D images are then transmitted (909) to the one or more second processing device(s) as a video stream. Then, the images are decoded (910) and displayed (911) at monitor(s) located in the treatment room(s) of the clinic.

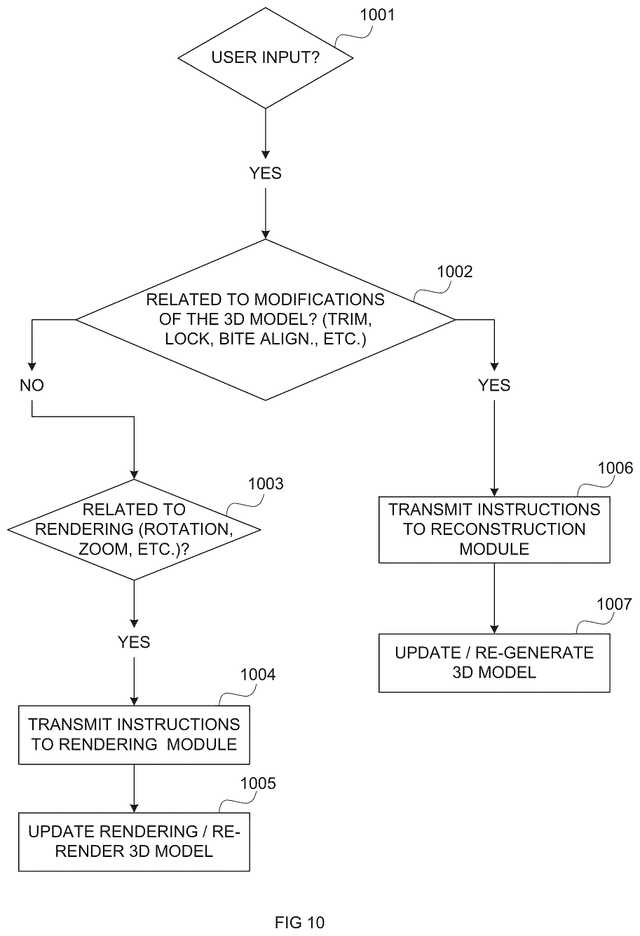

FIG. 10 shows a decision tree related to the methods disclosed herein. The decision tree relates to user input, which may be inputted via a graphical user interface, which is, in use, provided by the second computer program being executed by the second processing device. First, it is evaluated whether there is any user input (1001). If this is the case, it is evaluated if the user input relates to modifications of the 3D model such as trim, lock, bite align, etc. (1002). Such modifications need to be provided to the reconstruction module (1006), which is part of the first computer program. Accordingly, this type of user input may be specified and transmitted (1006) to the first computer program via an application programming interface (API). The first computer program is, in use, executed by the first processing device. Upon receipt of these instructions, the 3D model is updated and/or rebuilt (1007), preferably in real-time, whereby an updated 3D model can be displayed to the user. If the user input is not related to modifications of the 3D model, it is related to rendering (e.g. rotation of the model, zoom, pan, etc.) (1003), which means that such instructions should be provided to the rendering module (1004). The rendering module is part of the second computer program, which may be executed by either the first or second processing devices. Hence, in some embodiments, the 3D model is reconstructed remotely (e.g. in the cloud) but rendered locally (e.g. by a computer in the treatment room). Alternatively, in some embodiments, both the reconstruction and the rendering are performed remotely, e.g. by a remote server or a cloud-based server. The rendering module is configured to update the rendering or re-render the 3D model based on the received user input (1005). In this case, an updated 3D model is similarly displayed to the user, preferably in real-time.

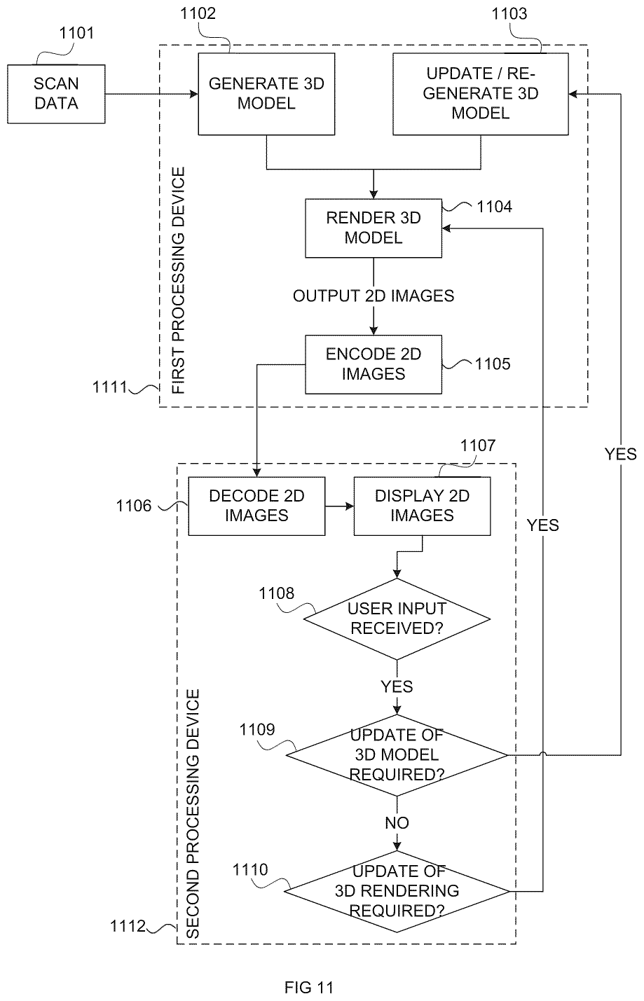

FIG. 11 shows an embodiment of a dental scanning system according to the present disclosure, wherein some of the key functions of the first (1111) and second processing devices (1112) are shown. In this embodiment, the first processing device is configured to both generate the 3D model (1102), render the 3D model (1104), and encode 2D images (1105) of the 3D model. The second processing device is configured to decode the 2D images (1106), display the 2D images (1107), and provide a graphical user interface for receiving user input (1108). Based on the assessment in step 1109 and/or step 1110, the 3D model is either re-generated (1103) or re-rendered (1104) by the first processing device (1111).

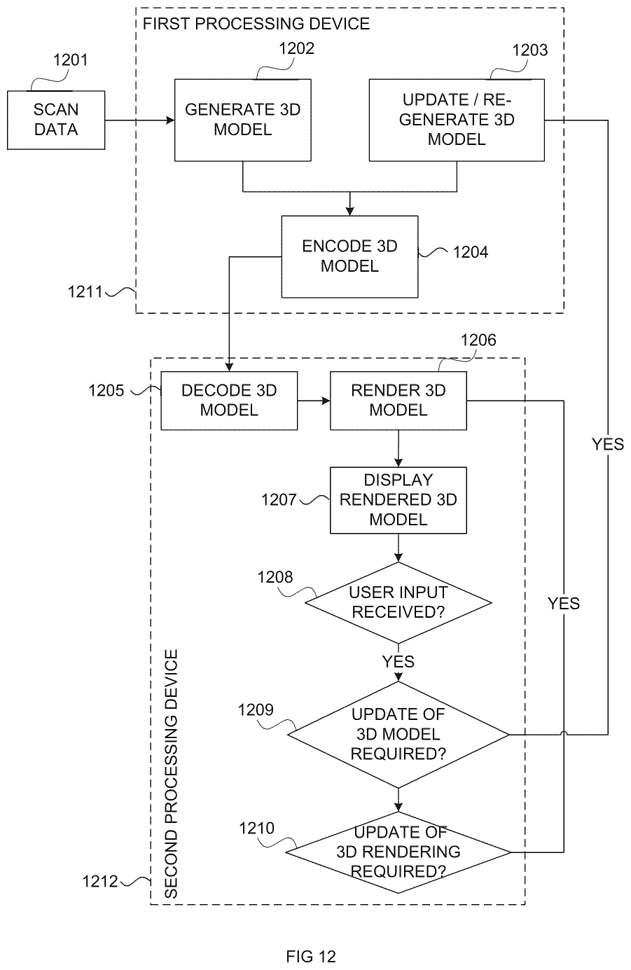

FIG. 12 shows an embodiment of a dental scanning system according to the present disclosure. In this embodiment, it is the 3D model itself which is encoded (1204) and transmitted to the second processing device(s), which are then configured to decode (1205) and render (1206) the 3D model and display the rendered model (1207). Accordingly, in this embodiment, the first processing device is configured to execute a first computer program, which, when executed, generates a 3D model (1202) or updates/regenerates a 3D model (1203) and then encodes the 3D model (1204). The second processing device is configured to execute a second computer program, which when executed, decodes the 3D model (1205), renders the 3D model (1206), and displays the rendered model (1207). The second computer program may further provide a graphical user interface for displaying the rendered model (1207) and receiving user input (1208).

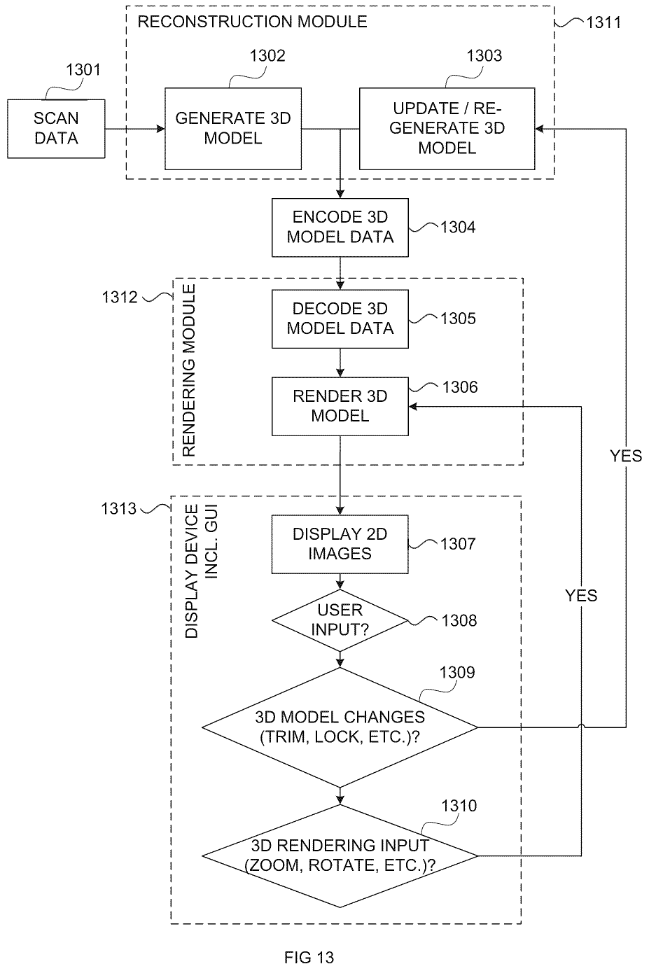

FIG. 13 shows essentially the same embodiment as the one shown in FIG. 12, however, here the functions are grouped not by processing device but by software module. Hence, the generation of the 3D model (1302) is performed by a reconstruction module (1311) which forms part of the first computer program, whereas the decoding (1305) and rendering (1306) is performed by a rendering module (1312) which forms part of a second computer program. The two computer programs may be executed by the same computer/processor or by two separate computers/processors. Alternatively, the software application may be split in two or more computer programs running on two or more computers.

FIG. 14 shows a schematic of different software functions and their interactions. The software functions may constitute microservices, i.e. forming a microservice architecture. The software functions may be provided or form part of one or more computer programs. Accordingly, the software functions may be split among several computer programs. As an example, a first computer program may comprise a first set of instructions, which when executed, performs the steps of reconstruction (1401) and rendering (1402). A second computer program may comprise a second set of instructions, which when executed, performs the steps of generating a user interface (1404) and displaying a 3D model (1405). In another example, a first computer program may comprise a first set of instructions, which when executed, performs the step of reconstruction a 3D model (1401). A second computer program may then comprise a second set of instructions, which when executed, performs the steps of rendering the 3D model (1402), generating a user interface (1404), and displaying the 3D model (1405). Other examples of arranging the software functions between one or more computer programs exist. The second computer program may further comprise instructions, which when executed, performs the step of receiving user input (1406) and/or interpreting user input in a model operations software function (1403). Alternatively, said functions may be provided by the execution of a third computer program. The first, second, third, etc. computer programs may be stored in one or more computer memories located on one or more computers or electronic devices. Accordingly, the first computer program may be stored on first computer memory on a first computer, and the second computer program may be stored on second computer memory on a second computer. Alternatively, the first and second computer programs may be stored on computer memory within the same computer. Accordingly, the entire software application may be designed as microservices, wherein the application comprises a plurality of independent software functions/services (1401, 1402, 1403, 1404, 1405, 1406, etc.) configured for generating/providing at least one type of output data and/or configured for receiving at least one type of input data. The microservices may be connected through application programming interfaces (indicated by arrows), which act as gateways between the different applications, allowing them to e.g. communicate, grant access, and/or transfer data to one another. The relations between the individual software functions (microservices) indicated by the arrows serve as an example and should not be construed as limiting. Accordingly, other relationships between the software functions may exist without departing from the scope of the invention. The microservice architecture of the software functions along with the application programming interfaces (APIs) is one example of implementing a software application, which is split between at least a first and a second computer program, said computer programs being configured to be executed by one or more computers/processing devices, preferably at least two computers. Other software applications may form part of a larger software ecosystem, which may be connected to the scanning software application. One configuration of executing one or more microservices on two or more processing devices is displayed in FIG. 1.

### Examples

In one example, illustrated in FIG. 1, a dental scanning system is provided in a dental clinic. In this example, the clinic is equipped with a wireless intraoral scanning device and a second processing device connected to a monitor. The monitor could be a display, a smart TV, a tablet computer, or other types of display devices. As an example, the monitor could be a display with a HDMI input socket and a dedicated dongle or box providing the processing and Wi-Fi functionality needed. The monitor is configured to display a rendered 3D reconstruction of scan data, i.e. a 3D model of the scanned object. The dental clinic is further equipped with a Wi-Fi router which is providing a Wireless Local Area Network (WLAN) in the clinic. The Wi-Fi router is further connected to the internet. All devices in the dental scanning system are connected to the WLAN directly or indirectly. In this example, the clinic does not need to have the first processing device present locally in the clinic. Instead, the first processing device is provided as a cloud-based service (comprising both cloud-based storage and cloud-based data processing), wherein said cloud-based service is accessible through an internet connection, e.g. established by the Wi-Fi router. In this example, cloud-based services refer to remotely installed servicers not physically present at the premises of the clinic.

One aspect of data processing is the generation of a virtual 3D representation of the physical object which is scanned by the scanning device. The generation of the virtual 3D representation (3D model) is performed by a scanning software application. The scanning software application is a component in a larger software ecosystem where information such as 3D data can be exchanged between different associated dental software applications (patient monitoring, design applications, manufacturing integrations, third party application, practice management systems etc.). The scanning software application may be split in two or more computer programs: A first computer program comprising a reconstruction module and a second computer program configured to control the integration to the surrounding software eco system, and further configured to provide a graphical user interface (GUI) for facilitating user interactions and displaying the virtual 3D model. The second computer program may further comprise a model operations tool module providing the user with possibilities to interact with the displayed virtual 3D model. The reconstruction module is capable of receiving patches of 3D information from the scanning device and performing alignment and stitching of the individual patches to obtain a fused 3D model. In addition to the reconstruction module, the first computer program comprises a rendering module which is capable of real-time rendering the fused 3D model, such that it is possible to display the 3D model in the graphical user interface while the model is being constructed during scanning.

When the data processing is performed by a cloud-based processing device, the scanning software application is preferably divided into two individual computer programs: A first computer program (reconstruction, running in the cloud) and the second computer program (front-end, running in the clinic). In this example, the first computer program is installed on the first processing device, here a cloud-based processer, which then may run on one or more high performance processor cores. The second computer program is installed on the second processing device directly associated with the display located in the dental clinic. The requirements to the processing capability and power of the second processing device are low, since no computational heavy tasks are required. The two separate computer programs may utilize Application programming interfaces (APIs) which are gateways between the different applications, allowing them to communicate, grant access, and transfer data to one another.

During a scanning session, where the scanning device is continuously generating scan data such as small individual patches of a patient's dentition, the scanning device may send the individual data patches directly to the first processing device via the internet connection. The scan patches are received by the first processing device, which is configured to perform alignment between the individual scan patches and fuse them together to construct a combined virtual 3D representation. During continuous addition of new scan data to existing data, the first processing device performs real-time rendering of the virtual 3D model. 2D images of the rendered virtual 3D model are continuously streamed via the internet from the first processing device into the user interface module running on the second processing device through the API. This enables the user to follow in real-time the continuous construction of the 3D model directly on the display in the clinic room.

If the scanning session is momentarily stopped, the reconstruction module preferably immediately sends complete surface information to a renderer component inside the second computer program. This enables the second computer program running on the second processing device to render and display the apparent state of the virtual 3D model. It further allows the dentist to perform model operations on the surface data via a GUI on the connected display. Such model operations could be spectating adjustments such as rotations, pan or zoom, or model editing operations like trim, lock, marked preparations, clearing the scan, manual bite alignment result, settings change such as adjust for contacts, and HD Photo, etc. All model operations are sent back to the first computer program running on the first processing device via the internet through the API to adjust the master data in the first computer program associated with the surface data manipulated in the user interface.

The scanning device may be configured to send data packages to both the first computer program running on the first processing device and directly to the second computer program running on the second processing device. Data packages sent to the first computer program may be 3D information, texture information such as infra-red images, fluorescence images, reflectance color images, x-ray images. Data packages send directly to the second computer program may be motion data for GUI navigation and/or 2D image preview data during a scanning session.

### List of Items

1. A dental scanning system for acquiring scan data of a physical three-dimensional dental object during a scanning session, the dental scanning system comprising:
   a scanning device comprising:
      one or more light projectors configured to generate an illumination pattern to be projected on a three-dimensional dental object during a scanning session; and
      one or more images sensors configured to acquire raw 2D images of the dental object in response to illuminating said object using the one or more light projectors;
   a processor configured to generate scan data by processing the raw 2D images, the scan data comprising depth information of the dental object;
   a first processing device configured to:
      receive the scan data and/or the raw 2D images from the scanning device e.g. via a wireless connection, a wired connection, or combinations thereof;
      generate a digital 3D model of at least part of the dental object based on the received scan data;
      optionally generate a plurality of digital 2D images of the digital 3D model;
      encode the digital 3D model and/or encode the digital 2D images in a video encoding format; and
      transmit the encoded images and/or the encoded 3D model to one or more second processing devices.
2. The system according to item 1, wherein the processor is part of the scanning device.
3. The system according to any of the preceding items, wherein the first processing device is further configured to generate a plurality of digital 2D images of the digital 3D model.
4. The system according to any of the preceding items, wherein the first processing device is configured to run a first computer program configured to generate and/or update the digital 3D model from the scan data.
5. The system according to any of the preceding items, wherein the first processing device is a remote server connected to the scanning device, wherein said connection is a wired connection, a wireless connection, and/or combinations thereof.
6. The system according to any of the preceding items, wherein the first processing device comprises one or more cloud-based processors, e.g. constituting a cloud-based processing cluster.
7. The system according to any of the preceding items, wherein the system further comprises a monitor for displaying a rendering of the digital 3D model and/or for displaying the digital 2D images.
8. The system according to item 7, wherein the monitor is connected to or integrated in the first and/or second processing device.
9. The system according to any of the preceding items, wherein the dental scanning system further comprises one or more second processing devices configured to:
   receive and decode the encoded images and/or the 3D model;
   optionally generate digital 2D images of the digital 3D model; and
   display the decoded images and/or the generated images on a monitor connected to or integrated in the second processing device(s).
10. The system according to item 9, wherein the second processing device(s) are configured to:
   receive and decode the digital 3D model;
   generate digital 2D images of the digital 3D model; and
   display the generated images on a monitor connected to or integrated in the second processing device(s).
11. The system according to any of the items 7-10, wherein the first and second processing device(s) are configured to connect to each other using a peer-to-peer connection.
12. The system according to any of the items 7-11, wherein the first and second processing device(s) are configured to establish the connection via a signaling server.
13. The system according to any of the items 11-12, wherein the peer-to-peer connection is a Web Real-Time Communication (WebRTC) connection.
14. The system according to any of the items 11-13, wherein the latency of the peer-to-peer connection is below 150 ms, preferably below 100 ms, even more preferably 75 ms.
15. The system according to any of the items 7-14, wherein the first and second processing device(s) are connected to each other via the internet and/or via one or more computer networks selected among the group of: local area network (LAN), wireless local area network (WLAN), wide area network (WAN), or combinations thereof.
16. The system according to any of the items 7-15, wherein the second processing device(s) are configured to run a second computer program providing a graphical user interface for receiving user input, the second computer program being configured to output the digital 2D images of the digital 3D model to the monitor.
17. The system according to any of the items 7-16, wherein the scanning device is configured to transmit the raw 2D images of the dental object to the second processing device(s).
18. The system according to any of the items 7-17, wherein the second processing device(s) are configured to receive the raw 2D image(s) from the scanning device, wherein the raw image(s) are used to provide a pre-view of the dental object in the graphical user interface of the second computer program.
19. The system according to any of the items 16-18, wherein the digital 3D model on the first processing device may be manipulated and/or updated through one or more user manipulations of the model via the graphical user interface of the second computer program running on the second processing device(s).
20. The system according to item 19, wherein the user manipulations are selected from the group of: rotate the model, zoom in/out on the model, change texture of the model, change colors of the model, and/or combinations thereof.
21. The system according to any of the items 19-20, wherein the user manipulations are selected from the group of: trim, lock, marked preparations, clearing the scan, manual bite alignment result, adjust for contacts, and/or combinations thereof.
22. The system according to any of the items 16-21, wherein the first and second computer programs are configured to communicate with each other via an application programming interface (API).
23. The system according to item 22, wherein the user manipulations are specified in the API.
24. The system according to any of the items 19-23, wherein the first processing device is configured to receive commands associated with the user manipulations via one or more application programming interface (API) calls.
25. The system according to any of the preceding items, wherein the dental scanning system further comprises a wireless network module configured to wirelessly connect the scanning device to a wireless local area network (WLAN).
26. The system according to any of the preceding items, wherein the scanning device further comprises a wireless network module configured to wirelessly connect the scanning device to a wireless local area network (WLAN).
27. The system according to any of the preceding items, wherein the dental scanning system further comprises a pod for holding the scanning device when it is not in use, wherein the pod comprises a wireless network module configured to wirelessly connect the scanning device to a wireless local area network (WLAN).
28. The system according to any of the preceding items, wherein the first and/or second processing device is connected to the scanning device via one or more LANs or WLANs.
29. The system according to any of the preceding items, wherein the first and/or second processing device is connected to the same WLAN as the scanning device.
30. The system according to any of the items 25-29, wherein the scanning device is configured to host a network access point for creating an initial connection to the first or second processing device.
31. The system according to item 30, wherein a monitor is connected to the first or second processing device and wherein a selection of one or more nearby scanning devices are presented on the monitor.
32. The system according to item 31, wherein the nearby scanning device(s) each host a network access point.
33. The system according to any of the items 31-32, wherein the wireless connection between the scanning device and the first and/or second processing device on the wireless local area network is established upon selecting a scanning device on the monitor.
34. The system according to any of the items 31-33, wherein the system is configured to display, on the monitor, only the scanning device having the highest signal strength, wherein the signal strength is associated with the network hosted by the scanning device.
35. The system according to any of the items 25-34, wherein the scanning device comprises a serial number, and wherein the wireless connection is established by transmitting or inputting the serial number to the first or second processing device.
36. The system according to item 35, wherein the system is configured to acquire an image of the serial number, e.g. using a camera connected to the dental scanning system, and wherein the wireless connection is established based on the serial number in the image.
37. The system according to any of the items 35-36, wherein the serial number is provided on a surface on the scanning device and/or wherein the serial number is provided as a QR code.
38. The system according to any of the items 35-37, wherein the scanning device is configured to transmit the scanner serial number to the first or second processing device using near-field communication (NFC).
39. The system according to any of the items 25-38, wherein the system is configured to transfer wireless network credentials using a software application running on an external electronic device, such as a smartphone or tablet.
40. The system according to item 39, wherein the external electronic device is configured for creating a bidirectional data link to the scanning device, e.g. based on Bluetooth.
41. The system according to any of items 7-40, wherein the second processing device(s) form a wired connection to the scanning device.
42. The system according to any of items 7-41, wherein the second processing device(s) are wirelessly connected to the scanning device via one or more WLANs.
43. The system according to any of items 7-42, wherein the second processing device(s) are connected to the same WLAN as the scanning device.
44. The system according to any of items 7-43, wherein the scanning device and the second processing device(s) are connected via one or more computer networks, such as one or more local area network(s) or wireless local area network(s), and wherein the first processing device is connected to the second processing device(s) via the internet.
45. The system according to any of items 7-44, wherein the scanning device and the second processing device(s) are connected via one or more LANs or WLANs, and wherein the first processing device is connected to the second processing device(s) via the internet.
46. The system according to any of items 7-45, wherein the monitor is an external monitor connected to the second processing device(s).
47. The system according to any of items 7-46, wherein the monitor is integrated in the second processing device(s).
48. The system according to any of the preceding items, wherein the scan data comprises raw digital 2D images.
49. The system according to any of the preceding items, wherein the first processing device is configured to output the digital 2D images to a display or a virtual display.
50. The system according to any of the preceding items, wherein the scanning device is a handheld intraoral scanner for acquiring images within an intraoral cavity of a subject during a scanning session.
51. The system according to any of the preceding items, wherein the three-dimensional dental object is an intraoral object of a subject, such as the teeth and gingiva of the subject.
52. The system according to any of the preceding items, wherein the scanning device comprises a processor configured to process the raw 2D images to generate a plurality of sub-scans, each of said sub-scans comprising depth data or a time stamp from which depth data can be inferred.
53. The system according to item 52, wherein each of the sub-scans further comprises texture information.
54. The system according to any of the preceding items, wherein the encoded 2D images are transmitted at a frame rate of at least 30 frames per second, preferably at least 60 frames per second.
55. The system according to any of the preceding items, wherein the first processing device is configured to continuously receive the scan data via a wireless network.
56. The system according to any of the preceding items, wherein the first processing device is configured to continuously receive the scan data via a wired connection.
57. The system according to any of the preceding items, wherein the first processing device is configured to continuously encode the digital 2D images and/or the scan data in a video encoding format.
58. The system according to item 57, wherein the video encoding format is selected among the group of: H.264, H.265, and VP8.
59. A method of transmitting digital images in real-time during a scanning session to one or more external processing devices, the method comprising the steps of:
   connect a scanning device to a wireless network, the scanning device being configured to acquire scan data from a three-dimensional dental object during a scanning session;
   continuously acquire scan data from the three-dimensional dental object during a scanning session using the scanning device, the scan data comprising a plurality of two-dimensional images and/or point clouds;
   continuously transmit the scan data to a first processing device via the wireless network;
   continuously generate and/or update a digital 3D model of at least part of the dental object based on the received scan data, wherein the generation of the digital 3D model is performed using the first processing device;
   continuously generate/render a plurality of digital 2D images of the digital 3D model using the first processing device;
   continuously encode the digital 2D images in a video encoding format using the first processing device;
   continuously transmit the encoded images to one or more second processing devices; and
   continuously decode and display the images in real-time using the one or more second processing devices.
60. A method of transmitting digital images in real-time during a scanning session to one or more external processing devices, the method comprising the steps of:
   connect a scanning device to a wireless network, the scanning device being configured to acquire scan data from a three-dimensional dental object during a scanning session;
   continuously acquire scan data from the three-dimensional dental object during a scanning session using the scanning device, the scan data comprising a plurality of two-dimensional images and/or point clouds;
   continuously transmit the scan data to a first processing device via the wireless network;
   continuously generate and/or update a digital 3D model of at least part of the dental object based on the received scan data, wherein the generation of the digital 3D model is performed using the first processing device;
   continuously encode the digital 3D model using the first processing device;
   continuously transmit the encoded digital 3D model to one or more second processing devices;
   continuously decode the encoded digital 3D model using the second processing device(s);
   continuously generate a plurality of digital 2D images of the digital 3D model using the second processing device(s); and
   continuously display the images in real-time using the second processing device(s).
61. The method according to any of the items 59-60, wherein the step of connecting the scanning device to the wireless network comprises the step of scanning a pattern on a display, wherein credentials of the wireless network are encoded in the pattern.
62. The method according to item 61, wherein the pattern is a QR code, a bar code, or a color code.
63. The method according to any of the preceding items, wherein the scanning device is configured to host a network access point.
64. The method according to any of the items 59-63, wherein the step of connecting the scanning device to the wireless network comprises the step of hosting a network access point from the scanning device.
65. The method according to any of the items 59-64, wherein the step of connecting the scanning device to the wireless network further comprises the step of selecting on a display connected to the first or second processing device, the scanning device among a list of nearby scanning devices.
66. The method according to item 65, wherein the list of nearby scanning devices is sorted by signal strength.
67. The method according to any of the items 59-66, wherein the scanning device having the highest signal strength is shown in a display connected to the first or second processing device, such that a user may connect to said scanning device by selecting it in the display.
68. The method according to any of the items 59-67, wherein the step of connecting the scanning device to the wireless network comprises the step of hosting a network access point from the scanning device and selecting on a display connected to the first or second processing device, the scanning device among a list of nearby scanning devices.
69. The method according to any of the items 59-68, wherein the scanning device is configured to connect to the first or second processing device via Bluetooth, and wherein a list of wireless networks is shown on a display connected to the first or second processing device, wherein the shown wireless networks are visible to the scanning device.
70. The method according to any of the items 59-69, wherein the step of connecting the scanning device to the wireless network comprises the step of authenticating the scanning device, wherein the authentication is based on a Bluetooth connection between the scanning device and the first or second processing device.
71. The method according to any of the items 59-70, wherein the step of connecting the scanning device to the wireless network comprises the step of capturing an image of a serial number located on the scanning device, wherein the connection is automatically established based on reading the serial number.
72. The method according to item 71, wherein the serial number is represented as a QR code.
73. The method according to any of the items 59-72, wherein the step of connecting the scanning device to the wireless network comprises the step of transferring wireless network credentials using a software application running on a smartphone or tablet.
74. The method according to any of the items 59-73, wherein the step of connecting the scanning device to the wireless network comprises the step of transmitting a serial number of the scanning device to the first or second processing device, wherein the transmission is based on near-field communication (NFC).
75. A method of generating a digital three-dimensional (3D) model of a dental object and displaying said 3D model remotely in real-time, the method comprising the steps of:
   receiving scan data of the dental object;
   reconstructing a digital three-dimensional (3D) model of at least part of the dental object based on the received scan data, wherein the reconstructing is performed by a first processing device;
   rendering a plurality of digital 2D images of the digital 3D model;
   encoding the digital 2D images in a video encoding format;
   transmitting the encoded 2D images to one or more second processing devices, wherein said second processing devices are located remotely from the first processing device; and
   decoding and displaying the 2D images, wherein the decoding and displaying is performed by the one or more second processing devices.
76. The method according to any of the items 59-61, wherein a peer-to-peer connection is established between the first and second processing device(s).
77. The method according to item 76, wherein the peer-to-peer connection is established using a signaling server.
78. The method according to any of the items 76-77, wherein the peer-to-peer connection is a Web Real-Time Communication (WebRTC) connection.
79. The method according to any of the items 76-78, wherein the latency of the peer-to-peer connection is below 150 ms, preferably below 100 ms, even more preferably 75 ms.
80. The method according to any of the items 59-79, wherein the video encoding format is selected among the group of: H.264, H.265, and VP8.
81. The method according to any of the items 59-80, wherein the encoded images or the encoded 3D model is transmitted from the first processing device to the second processing device(s) at a bitrate of between approximately 4.8 Mbps to approximately 8.8 Mbps.
82. The method according to any of the items 59-81, wherein the digital 2D images are rendered using Simple DirectMedia Layer (SDL).
83. The method according to any of the items 59-82, wherein the method is performed using the dental scanning system according to any of the items 1-58.
84. A system for displaying images of a digital three-dimensional (3D) model of a dental object, wherein the system comprises:
   a first processing device comprising a processor configured to execute machine-readable instructions such that when the machine-readable instructions are executed by the processor, the first processing device is caused to perform:
      receiving scan data of a three-dimensional dental object;
      reconstructing a digital three-dimensional (3D) model of at least part of the dental object based on the received scan data;
      rendering a plurality of digital images of the digital 3D model;
      encoding the digital images in a video encoding format;
      transmitting the encoded images to one or more second processing devices, wherein said second processing devices are located remotely from the first processing device;
   one or more second processing devices, each comprising a processor configured to execute machine-readable instructions such that when the machine-readable instructions are executed by the processor, the second processing device(s) are caused to perform:
      decoding the images;
      displaying the images.
85. A system comprising:
   a scanning device comprising:
      means for connecting the scanning device to a computer network;
      means for continuously acquiring scan data from a three-dimensional dental object during a scanning session;
      means for continuously transmitting the scan data to a first processing device via the computer network;
   a first processing device comprising:
      means for continuously receiving the scan data via the computer network;
      means for continuously generating a digital 3D model of at least part of the dental object based on the received scan data;
      means for continuously encoding the 3D model or data associated with the 3D model;
      means for continuously transmitting the 3D model and/or data;
   one or more second processing devices comprising:
      means for continuously generating a plurality of digital 2D images of the digital 3D model; and
      means for continuously displaying the images in real-time using the one or more second processing devices.
86. A system comprising:
   a scanning device comprising:
      means for connecting the scanning device to a computer network;
      means for continuously acquiring scan data from a three-dimensional dental object during a scanning session;
      means for continuously transmitting the scan data to a first processing device via the computer network;
   a first processing device comprising:
      means for continuously receiving the scan data via the computer network;
      means for continuously generating a digital 3D model of at least part of the dental object based on the received scan data;
      means for continuously generating a plurality of digital 2D images of the digital 3D model;
      means for continuously encoding the digital 2D images in a video encoding format;
      means for continuously transmitting the encoded images to one or more second processing devices; and
   one or more second processing devices comprising:
      means for continuously decoding and displaying the images in real-time using the one or more second processing devices.
87. The system according to item 86, wherein the computer network is a wireless network and wherein the means for connecting the scanning device to a computer network is a Wi-Fi module.
88. The system according to any of the items 86-87, wherein the means for continuously acquiring scan data comprises at least one light projector for generating an illumination pattern and at least one image sensor for acquiring images of the dental object during the scanning session.
89. The system according to any of the items 86-88, wherein the means for continuously receiving the scan data comprises a wireless network interface controller.
90. The system according to any of the items 84-89, wherein the first processing device comprises a first computer program comprising instructions which, when executed by the first processing device, causes the first processing device to perform the steps of:
   generating a digital 3D model of at least part of the dental object based on the received scan data;
   generating a plurality of digital 2D images of the digital 3D model;
   encoding the digital 2D images in a video encoding format;
   transmitting the encoded images to one or more second processing devices.
91. The system according to any of the items 84-90, wherein the second processing device(s) comprises a second computer program comprising instructions which, when executed by the second processing device(s), causes the second processing device(s) to perform the steps of:
   receiving and decoding the digital 2D images;
   providing a graphical user interface (GUI) for receiving user input; and
   displaying the 2D image(s) along with the GUI.
92. The system according to item 91, wherein the second processing device(s), when the second computer program is executed, is further caused to perform the steps of:
   receiving user input via the GUI and transmitting the input to the first processing device.
93. The system according to item 92, wherein the user input is selected among the group of: zoom, pan, rotate, change of texture, change of colors, trim, lock, marked preparations, clearing the scan, manual bite alignment result, adjust for contacts, and/or combinations thereof.

Although some embodiments have been described and shown in detail, the disclosure is not restricted to such details, but may also be embodied in other ways within the scope of the subject matter defined in the following claims. In particular, it is to be understood that other embodiments may be utilized and structural and functional modifications may be made without departing from the scope of the present invention. Furthermore, the skilled person would find it apparent that unless an embodiment is specifically presented only as an alternative, different disclosed embodiments may be combined to achieve a specific implementation and such specific implementation is within the scope of the disclosure.

A claim may refer to any of the preceding claims, and “any” is understood to mean “any one or more” of the preceding claims.

It should be emphasized that the term “comprises/comprising/including” when used in this specification is taken to specify the presence of stated features, integers, operations, steps or components but does not preclude the presence or addition of one or more other features, integers, steps, components or groups thereof.

In claims enumerating several means, several of these means can be embodied by one and the same item of hardware. The mere fact that certain measures are recited in mutually different dependent claims or described in different embodiments does not indicate that a combination of these measures cannot be used to advantage.

1. A dental scanning system for acquiring scan data of a physical three-dimensional dental object during a scanning session, the dental scanning system comprising:
a scanning device, wherein the scanning device is a handheld intraoral scanner for acquiring images within an intraoral cavity of a subject during the scanning session, said scanning device comprising:
one or more light projectors configured to generate an illumination pattern to be projected on a three-dimensional dental object during a scanning session; and
one or more images sensors configured to acquire raw 2D images of the dental object in response to illuminating said object using the one or more light projectors;
a processor configured to generate scan data by processing the raw 2D images, the scan data comprising depth information of the dental object;
a first processing device configured to:
receive the scan data and/or receive the raw 2D images from the scanning device and subsequently generate scan data by processing the raw 2D images;
generate a digital 3D model of at least part of the dental object based on the scan data;
generate a plurality of digital 2D images of the digital 3D model;
encode the digital 2D images in a video encoding format; and
transmit the encoded images to one or more second processing devices;
one or more second processing devices configured to:
receive and decode the encoded images; and
display the decoded images on a monitor connected to or integrated in the second processing device(s).

2. The dental scanning system according to claim 1, wherein the first processing device is a remote server connected to the scanning device, wherein said connection is a wired connection, a wireless connection, and/or combinations thereof.

3. The dental scanning system according to claim 1, wherein the first processing device is provided as a cloud-based service accessible through an internet connection.

4. The dental scanning system according to claim 1, wherein the first and second processing device(s) are configured to connect to each other using a peer-to-peer connection.

5. The dental scanning system according to claim 4, wherein the latency of the peer-to-peer connection is below 150 ms.

6. The dental scanning system according to claim 1, wherein the second processing device(s) are configured to run a second computer program comprising instructions which, when the program is executed by the second processing device(s), cause the second processing device(s) to carry out the step of generating a graphical user interface for receiving user input.

7. The dental scanning system according to claim 6, wherein the decoded images are displayed within the graphical user interface provided by the second processing device(s).

8. The dental scanning system according to claim 6, wherein the first processing device is configured to run a first computer program comprising instructions which, when the program is executed by the first processing device(s), cause the first processing device(s) to carry out the step of generating or updating the digital 3D model from the scan data, wherein the first and second computer programs are configured to communicate with each other via an application programming interface (API).

9. The dental scanning system according to claim 6, wherein the digital 3D model on the first processing device may be manipulated or updated through one or more user manipulations of the model via the graphical user interface.

10. The dental scanning system according to claim Error! Reference source not found., wherein the user manipulations include operations that change the digital 3D representation, wherein the first processing device is configured to re-generate the digital 3D representation based on said manipulations/changes.

11. The dental scanning system according to claim 9, wherein the first processing device is configured to receive commands associated with the user manipulations via one or more application programming interface (API) calls.

12. The dental scanning system according to claim 1, wherein the scanning device further comprises a wireless network module configured to wirelessly connect the scanning device to a wireless local area network (WLAN).

13. The dental scanning system according to claim 1, wherein the scanning device is configured to establish a Bluetooth connection between the scanning device and the first or second processing device.

14. The dental scanning system according to claim 13, wherein the dental scanning system is configured to connect the scanning device to a wireless local area network by transferring network credentials associated with said wireless network via the Bluetooth connection.

15. The dental scanning system according to claim 1, wherein the scanning device and the second processing device(s) are connected via one or more computer networks, such as one or more local area network(s) or wireless local area network(s), and wherein the first processing device is connected to the second processing device(s) via the internet.

16. The dental scanning system according to claim 1, wherein the video encoding format is selected among the group of: H.264, H.265, and VP8.

17. The dental scanning system according to claim 1, wherein the encoded 2D images are transmitted at a frame rate of at least 30 frames per second.

18. A method of transmitting digital images in real-time during a scanning session to one or more external processing devices, the method comprising the steps of:
connecting a scanning device to a wireless network, the scanning device being configured to acquire scan data from a three-dimensional dental object during a scanning session;
continuously acquiring scan data from the three-dimensional dental object during a scanning session using the scanning device, the scan data comprising a plurality of two-dimensional images;
continuously transmitting the scan data to a first processing device via the wireless network;
continuously generating and/or updating a digital 3D model of at least part of the dental object based on the received scan data, wherein the generation of the digital 3D model is performed by the first processing device;
continuously generating a plurality of digital 2D images of the digital 3D model using the first processing device;
continuously encoding the digital 2D images in a video encoding format using the first processing device;
continuously transmitting the encoded images to one or more second processing devices; and
continuously decoding and displaying the images in real-time using the one or more second processing devices.

19. The method according to claim 18, wherein the step of connecting the scanning device comprises the step of establishing a Bluetooth connection between the scanning device and an electronic device.

20. The method according to claim 19, wherein network credentials and/or a network certificate associated with the wireless network is transferred to the scanning device via the Bluetooth connection, whereby the scanning device is connected to the wireless network.

21. The method according to claim 19, wherein the electronic device is the first or second processing device.

22. The method according to claim 18, wherein the step of connecting the scanning device to the wireless network comprises the step of displaying a list of Wi-Fi networks visible by the scanning device on a monitor.

23. The method according to claim 18, wherein the step of connecting the scanning device to the wireless network comprises the steps of hosting a network access point from the scanning device, and selecting on a display connected to the first or second processing device, the scanning device.

24. The method according to claim 18, wherein the method is performed by a dental scanning system for acquiring scan data of a physical three-dimensional dental object during a scanning session, the dental scanning system comprising:
a scanning device, wherein the scanning device is a handheld intraoral scanner for acquiring images within an intraoral cavity of a subject during the scanning session, said scanning device comprising:
one or more light projectors configured to generate an illumination pattern to be projected on a three-dimensional dental object during a scanning session; and
one or more images sensors configured to acquire raw 2D images of the dental object in response to illuminating said object using the one or more light projectors;
a processor configured to generate scan data by processing the raw 2D images, the scan data comprising depth information of the dental object;
a first processing device configured to:
receive the scan data and/or receive the raw 2D images from the scanning device and subsequently generate scan data by processing the raw 2D images;
generate a digital 3D model of at least part of the dental object based on the scan data;
generate a plurality of digital 2D images of the digital 3D model;
encode the digital 2D images in a video encoding format; and
transmit the encoded images to one or more second processing devices;
one or more second processing devices configured to:
receive and decode the encoded images; and
display the decoded images on a monitor connected to or integrated in the second processing device(s).
